(19) United States
(12) Patent Application Publication
(10) Pub. No.: US 2023/0100137 A1
(43) Pub. Date: Mar. 30, 2023

(54) METHODS OF TREATING AND PREVENTING ALLOANTIBODY DRIVEN CHRONIC GRAFT VERSUS HOST DISEASE

(71) Applicants: Pharmacyclics LLC, North Chicago, IL (US); Regents of the University of Minnesota, Minneapolis, MN (US)

(72) Inventors: Bruce R. Blazar, Golden Valley, MN (US); Ryan Flynn, Minneapolis, MN (US)

(21) Appl. No.: 17/733,419

(22) Filed: Apr. 29, 2022

Related U.S. Application Data
(63) Continuation of application No. 16/942,151, filed on Jul. 29, 2020, now abandoned.
Application No. 16/942,151 is a continuation of application No. 15/829,087, filed on Dec. 1, 2017, now abandoned.
Application No. 15/829,087 is a continuation of application No. 14/558,297, filed on Dec. 2, 2014, now abandoned.
(60) Provisional application No. 61/973,178, filed on Mar. 31, 2014.
(60) Provisional application No. 61/910,944, filed on Dec. 2, 2013.

(51) Int. Cl. A61K 31/519; A61K 35/28; A61K 45/06

(52) U.S. Cl. CPC A61K 31/519; A61K 35/28; A61K 45/06; A61K 2035/124

(57) ABSTRACT

Described herein are methods for treating and preventing alloantibody driven chronic graft versus host disease (cGVHD). The methods include administering to an individual in need thereof ibrutinib for treating and preventing alloantibody driven graft versus host disease.

### CROSS-REFERENCE

This application claims the benefit of U.S. Provisional Application No. 61/973,178, filed Mar. 31, 2014; and U.S. Provisional Application No. 61/910,944, filed Dec. 2, 2013, which applications are incorporated herein by reference.

### STATEMENT AS TO FEDERALLY SPONSORED RESEARCH

The invention disclosed herein was made, at least in part, with U.S. government support under Grant No. P01 CA142106 by the National Institutes of Health. Accordingly, the U.S. Government has certain rights in this invention.

### SEQUENCE LISTING

The instant application contains a Sequence Listing which has been submitted in ASCII format via EFS-Web and is hereby incorporated by reference in its entirety. Said ASCII copy, created on Dec. 1, 2014, is named 25922-301-201SEQ.txt and is 629 bytes in size.

### BACKGROUND

Chronic graft versus host disease (cGVHD) is the most common long-term complication following allogeneic stem cell transplant (SCT), affecting 30-70% of patients who survive beyond the first 100 days. cGVHD and its associated immune deficiency has been identified as a leading cause of non-relapse mortality (NRM) in allogeneic SCT survivors. SCT survivors with cGVHD are 4.7 times as likely to develop severe or life-threatening health conditions compared with healthy siblings, and patients with active cGVHD are more likely to report adverse general health, mental health, functional impairments, activity limitation, and pain than allo-SCT survivors with no history of cGVHD. Any organ system can be affected, and further morbidity is frequently caused by long-term exposure to the corticosteroids and calcineurin inhibitors required to treat the condition. Alloreactive B-cells in addition to specific CD4 T-cell subsets are key mediators of cGVHD. B-cells and pathogenic alloantibody deposition are aberrantly hyperactive in human cGVHD.

### SUMMARY OF THE INVENTION

Disclosed herein, in some embodiments, are methods of treating alloantibody driven chronic graft versus host disease (cGVHD) in a patient in need thereof, comprising administering a therapeutically effective amount of an ACK inhibitor (e.g., an ITK or BTK inhibitor). In some embodiments, there are provided methods of treating alloantibody driven chronic graft versus host disease (cGVHD) in a patient, comprising administering to a patient in need thereof a therapeutically effective amount of a compound of Formula (A) having the structure:

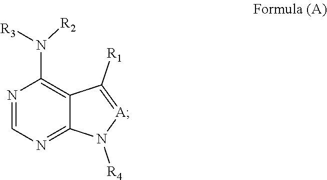

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

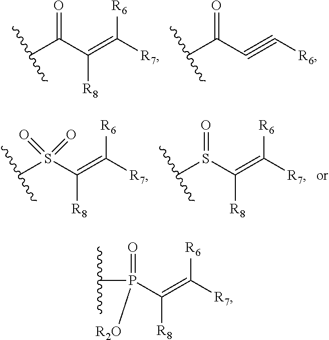

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof, thereby treating the cGVHD in the patient. In some embodiments, L₃, X and L₄ taken together form a nitrogen containing heterocyclic ring. In some embodiments, the nitrogen containing heterocyclic ring is a piperidine group. In some embodiments, G is

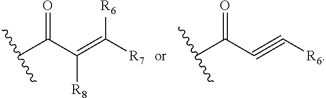

some embodiments, the compound of Formula (A) is (R)-1-(3-(4-amino-3-(4-phenoxyphenyl)-1H-pyrazolo[3,4-d]pyrimidin-1-yl)piperidin-1-yl)prop-2-en-1-one (ibrutinib)

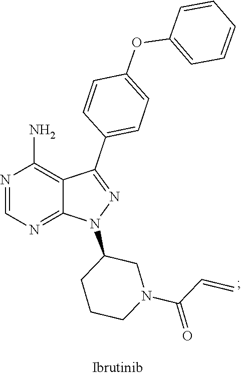 [SMILES: C=CC(=O)N1CCC[C@@H](n2nc(-c3ccc(Oc4ccccc4)cc3)c3c(N)ncnc32)C1]

or a pharmaceutically acceptable salt thereof. In some embodiments, the patient exhibits one or more symptoms of cGVHD. In some embodiments, the cGVHD is treatment naive cGVHD. In some embodiments, the cGVHD is non-sclerodermatous cGVHD. In some embodiments, the cGVHD is multi-organ cGVHD. In some embodiments, the cGVHD is bronchiolitis obliterans syndrome. In some embodiments, the cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, fibrosis is reduced. In some embodiments, lung fibrosis is reduced. In some embodiments, liver fibrosis is reduced. In some embodiments, immunoglobulin (Ig) deposition in tissue is reduced. In some embodiments, the patient has cancer. In some embodiments, the patient has a hematological malignancy. In some embodiments, the patient has a relapsed or refractory hematological malignancy. In some embodiments, the patient has a B-cell malignancy. In some embodiments, the patient has a T-cell malignancy. In some embodiments, the patient has a leukemia, a lymphoma, or a myeloma. In some embodiments, the B-cell malignancy is a non-Hodgkin's lymphoma. In some embodiments, the B-cell malignancy is chronic lymphocytic leukemia (CLL). In some embodiments, the B-cell malignancy is a relapsed or refractory B-cell malignancy. In some embodiments, the B-cell malignancy is a relapsed or refractory non-Hodgkin's lymphoma. In some embodiments, the B-cell malignancy is a relapsed or refractory CLL. In some embodiments, the patient has high risk CLL. In some embodiments, the patient has a 17p chromosomal deletion. In some embodiments, the patient has 10%, 20%, 30%, 40%, 50%, 60%, 70%, 80%, 90%, or greater CLL as determined by bone marrow biopsy. In some embodiments, the patient has received one or more prior anticancer agents. In some embodiments, the patient has received a cell transplantation. In some embodiments, the cell transplantation is a hematopoietic cell transplantation. In some embodiments, the cell transplantation is an allogeneic bone marrow or hematopoietic stem cell transplant. In some embodiments, the compound of Formula (A) is administered concurrently with an allogeneic bone marrow or hematopoietic stem cell transplant. In some embodiments, the compound of Formula (A) is administered subsequent to an allogeneic bone marrow or hematopoietic stem cell transplant. In some embodiments, the amount of the ACK inhibitor compound (e.g., a compound of Formula (A)) prevents or reduces cGVHD while maintaining a graft-versus-leukemia (GVL) reaction effective to reduce or eliminate the number of cancerous cells in the blood of the patient. In some embodiments, the compound of Formula (A) is administered at a dosage of between about 0.1 mg/kg per day to about 100 mg/kg per day. In some embodiments, the amount of the compound of Formula (A) administered is about 40 mg/day, about 140 mg/day, about 420 mg/day, about 560 mg/day, or about 840 mg/day. In some embodiments, the compound of Formula (A) is administered from day 1 to about day 1000 following allogeneic bone marrow or hematopoietic stem cell transplant. In some embodiments, the compound of Formula (A) is administered from the onset of alloantibody driven cGVHD symptoms to about day 1000 following allogeneic bone marrow or hematopoietic stem cell transplant. In some embodiments, the compound of Formula (A) is administered orally. In some embodiments, the compound of Formula (A) is administered in combination with one or more additional therapeutic agents.

In some embodiments, disclosed herein is a method of preventing the occurrence of alloantibody driven chronic graft versus host disease (cGVHD) or reducing the severity of alloantibody driven cGVHD occurrence in a patient requiring cell transplantation, comprising administering a therapeutically effective amount of an ACK inhibitor (e.g., an ITK or BTK inhibitor). In some embodiments, disclosed herein is a method of preventing the occurrence of alloantibody driven chronic graft versus host disease (cGVHD) or reducing the severity of alloantibody driven cGVHD occurrence in a patient requiring cell transplantation, comprising administering a therapeutically effective amount of a compound of Formula (A) having the structure:

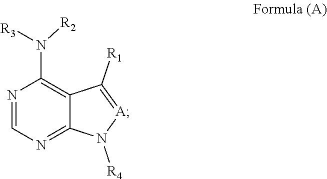

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

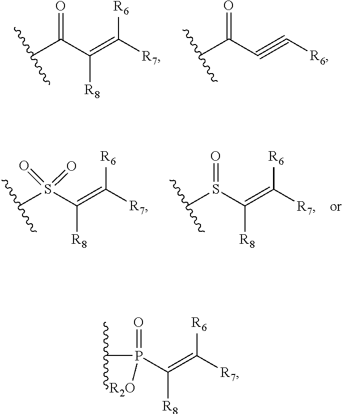

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

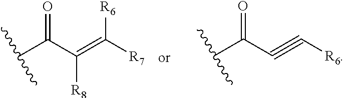

In some embodiments, disclosed herein is a method of preventing the occurrence of alloantibody driven chronic graft versus host disease (cGVHD) or reducing the severity of alloantibody driven cGVHD occurrence in a patient requiring cell transplantation, comprising administering a therapeutically effective amount of an ACK inhibitor (e.g., an ITK or BTK inhibitor). In some embodiments, disclosed herein is a method of preventing the occurrence of alloantibody driven chronic graft versus host disease (cGVHD) or reducing the severity of alloantibody driven cGVHD occurrence in a patient requiring cell transplantation, comprising administering a therapeutically effective amount of a compound of Formula (A):

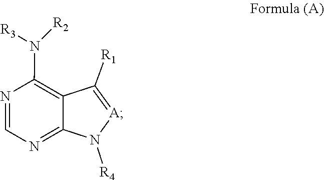

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

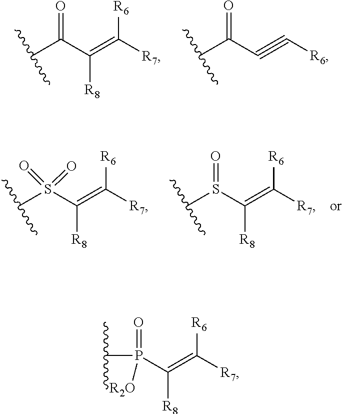

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof, is administered prior to or concurrently with the allogeneic hematopoietic stem cells and/or allogeneic T-cells. In some embodiments, L₃, X and L₄ taken together form a nitrogen containing heterocyclic ring. In some embodiments, the nitrogen containing heterocyclic ring is a piperidine group. In some embodiments, G is

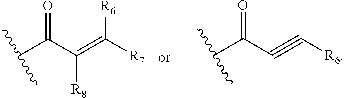

In some embodiments, disclosed herein is a method of preventing the occurrence of alloantibody driven chronic graft versus host disease (cGVHD) or reducing the severity of alloantibody driven cGVHD occurrence in a patient requiring cell transplantation, comprising administering a therapeutically effective amount of (R)-1-(3-(4-amino-3-(4-phenoxyphenyl)-1H-pyrazolo[3,4-d]pyrimidin-1-yl)piperidin-1-yl)prop-2-en-1-one (ibrutinib)

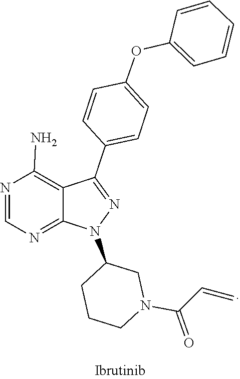 [SMILES: C=CC(=O)N1CCC[C@@H](n2nc(-c3ccc(Oc4ccccc4)cc3)c3c(N)ncnc32)C1]

In some embodiments the alloantibody driven cGVHD is non-sclerodermatous cGVHD. In some embodiments the alloantibody driven cGVHD is multi-organ cGVHD. In some embodiments the alloantibody driven cGVHD is bronchiolitis obliterans syndrome. In some embodiments, the alloantibody driven cGVHD is lung cGVHD. In some embodiments, the patient has cancer. In some embodiments, the patient has a hematological malignancy. In some embodiments, the patient has a B-cell malignancy. In some embodiments, the cell transplantation is a hematopoietic cell transplantation. In some embodiments, the patient has or will receive an allogeneic bone marrow or hematopoietic stem cell transplant. In some embodiments, ibrutinib is administered concurrently with an allogeneic bone marrow or hematopoietic stem cell transplant. In some embodiments, ibrutinib is administered prior to an allogeneic bone marrow or hematopoietic stem cell transplant.

Disclosed herein, in some embodiments, is a method of treating a patient for alleviation of an alloantibody response, with alleviation of consequently developed chronic graft versus host disease (cGVHD), comprising administering to the patient allogeneic hematopoietic stem cells and/or allogeneic T-cells, wherein a therapeutically effective amount of an ACK inhibitor (e.g., an ITK or BTK inhibitor). Disclosed herein, in some embodiments, is a method of treating a patient for alleviation of an alloantibody response, with alleviation of consequently developed chronic graft versus host disease (cGVHD), comprising administering to the patient allogeneic hematopoietic stem cells and/or allogeneic T-cells, and a therapeutically effective amount of a compound of Formula (A).

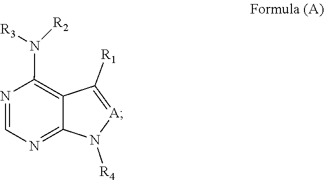

wherein.

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

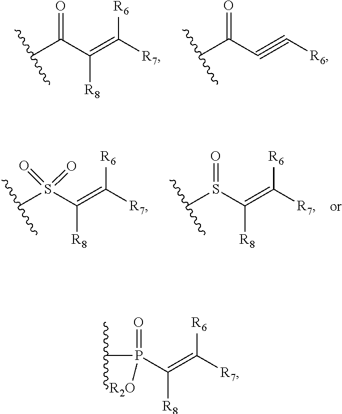

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. Disclosed herein, in some embodiments, is a method of treating a patient for alleviation of an alloantibody response, with alleviation of consequently developed chronic graft versus host disease (cGVHD), comprising administering to the patient allogeneic hematopoietic stem cells and/or allogeneic T-cells, and a therapeutically effective amount of (R)-1-(3-(4-amino-3-(4-phenoxyphenyl)-1H-pyrazolo[3,4-d]pyrimidin-1-yl)piperidin-1-yl)prop-2-en-1-one (ibrutinib)

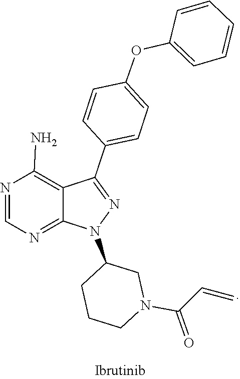 [SMILES: C=CC(=O)N1CCC[C@@H](n2nc(-c3ccc(Oc4ccccc4)cc3)c3c(N)ncnc32)C1]

In some embodiments the alloantibody driven cGVHD is non-sclerodermatous cGVHD. In some embodiments the alloantibody driven cGVHD is multi-organ cGVHD. In some embodiments the alloantibody driven cGVHD is bronchiolitis obliterans syndrome. In some embodiments, the alloantibody driven cGVHD is lung cGVHD. In some embodiments, the patient has cancer. In some embodiments, the patient has a hematological malignancy. In some embodiments, the patient has a B-cell malignancy. In some embodiments, the cell transplantation is a hematopoietic cell transplantation. In some embodiments, the patient has or will receive an allogeneic bone marrow or hematopoietic stem cell transplant. In some embodiments, ibrutinib is administered concurrently with an allogeneic bone marrow or hematopoietic stem cell transplant. In some embodiments, ibrutinib is administered prior to an allogeneic bone marrow or hematopoietic stem cell transplant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 exemplifies collagen deposition and pulmonary function are therapeutically improved in a murine model of allo-HSCT induced cGVHD with bronchiolitis obliterans. A-C) PFTs were performed at day 60 post-transplant on anesthetized animals. Animals were artificially ventilated and A) resistance, B) elastance, and C) compliance were measured as parameters of distress in lung function in animals receiving low-dose splenocytes (S) in addition to bone marrow (BM). Error bars=s.e.m. D and E) Collagen deposition within pulmonary tissues was determined with a Masson trichrome staining kit; blue indicates collagen deposition. D) Representative images of collagen deposition observed in each treatment cohort. Blue staining represents Masson Trichrome stained collagen. E) Quantification of collaged deposition as a ratio of blue area to total area of tissue was performed with the analysis tool in Photoshop CS3.

FIG. 2 exemplifies survival of cGVHD mice in C57BL/6→B10.BR model. Kaplan Meier plot of overall survival for bone marrow (BM) non-cGVHD mice, BM+splenocyte (S) engrafted cGVHD irrelevant vehicle treated mice, or Ibrutinib treated BM+S engrafted mice.

FIG. 3 exemplifies body weight of cGVHD mice in C57BL/6→B10.BR model. Bodyweight measurements for bone marrow (BM) non-cGVHD mice, BM+splenocyte (S) engrafted cGVHD irrelevant vehicle treated mice, or Ibrutinib treated BM+S engrafted mice.

FIG. 4 exemplifies germinal center reactions and pulmonary immunoglobulin deposition are therapeutically abated with administration of ibrutinib. A) Germinal centers were imaged by staining 6 um spleen sections with PNA conjugated to rhodamine. B) Splenocytes were purified from transplanted mice on day 60 and frequency of germinal center B cells were quantified. C) 6 μm lung sections from day 60 transplanted mice were stained with anti-mouse Ig conjugated to FITC. D) quantified with Adobe Photoshop CS3.

FIG. 5 exemplifies expression of BTK in donor-derived B cells is necessary for the development of BO. A) Day 60 pulmonary function tests from mice transplanted with low levels of WT T-cells and either WT or XID (kinase inactive BTK) bone marrow. B and C) Pathology of lung, liver, and spleen of day 60 transplanted mice. n=5 mice/group from 2 independent experiments.

FIG. 6 exemplifies development of BO is dependent on ITK expression in donor mature T cells. A) Day 60 pulmonary function tests mice transplanted with WT bone marrow and low numbers of either WT T-cells or ITK deficient T cells. B and C) Pathologic scores in lung, liver and spleen of day 60 transplanted mice. n=5 mice/group from 2 independent experiments.

### DETAILED DESCRIPTION OF THE INVENTION

Disclosed herein, in some embodiments, are methods of treating alloantibody driven chronic graft versus host disease (cGVHD) in a patient in need thereof, comprising administering a therapeutically effective amount of an ACK inhibitor (e.g., an ITK or BTK inhibitor). In some embodiments, there are provided methods of treating alloantibody driven chronic graft versus host disease (cGVHD) in a patient, comprising administering to a patient in need thereof a therapeutically effective amount of a compound of Formula (A) having the structure:

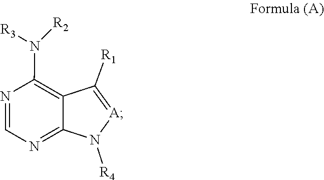

wherein:
A is N;
R₁ is phenyl-O-phenyl or phenyl-S-phenyl;
R₂ and R₃ are independently H;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;
G is

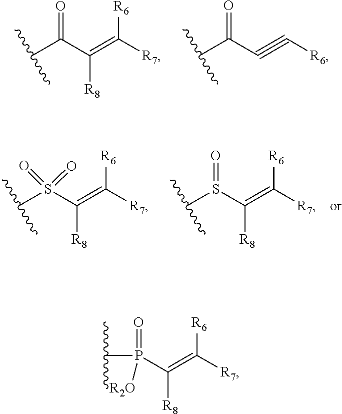

wherein,
R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or substituted or unsubstituted alkyl; or
a pharmaceutically acceptable salt thereof, thereby treating the cGVHD in the patient. In some embodiments, L₃, X and L₄ taken together form a nitrogen containing heterocyclic ring. In some embodiments, the nitrogen containing heterocyclic ring is a piperidine group. In some embodiments, G is

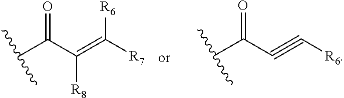

In some embodiments, the compound of Formula (A) is (R)-1-(3-(4-amino-3-(4-phenoxyphenyl)-1H-pyrazolo[3,4-d]pyrimidin-1-yl)piperidin-1-yl)prop-2-en-1-one (ibrutinib)

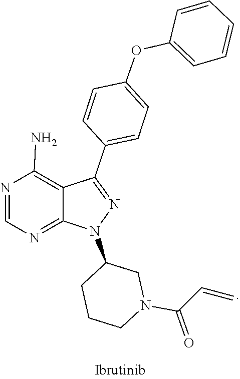 [SMILES: C=CC(=O)N1CCC[C@@H](n2nc(-c3ccc(Oc4ccccc4)cc3)c3c(N)ncnc32)C1]

or a pharmaceutically acceptable salt thereof. In some embodiments, the patient exhibits one or more symptoms of alloantibody driven cGVHD. In some embodiments, the alloantibody driven cGVHD is treatment naive cGVHD. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, fibrosis is reduced. In some embodiments, lung fibrosis is reduced. In some embodiments, liver fibrosis is reduced. In some embodiments, immunoglobulin (Ig) deposition in tissue is reduced. In some embodiments, the patient has cancer. In some embodiments, the patient has a hematological malignancy. In some embodiments, the patient has a relapsed or refractory hematological malignancy. In some embodiments, the patient has a B-cell malignancy. In some embodiments, the patient has a T-cell malignancy. In some embodiments, the patient has a leukemia, a lymphoma, or a myeloma. In some embodiments, the B-cell malignancy is a non-Hodgkin's lymphoma. In some embodiments, the B-cell malignancy is chronic lymphocytic leukemia (CLL). In some embodiments, the B-cell malignancy is a relapsed or refractory B-cell malignancy. In some embodiments, the B-cell malignancy is a relapsed or refractory non-Hodgkin's lymphoma. In some embodiments, the B-cell malignancy is a relapsed or refractory CLL. In some embodiments, the patient has high risk CLL. In some embodiments, the patient has a 17p chromosomal deletion. In some embodiments, the patient has 10%, 20%, 30%, 40%, 50%, 60%, 70%, 80%, 90%, or greater CLL as determined by bone marrow biopsy. In some embodiments, the patient has received one or more prior anticancer agents. In some embodiments, the patient has received a cell transplantation. In some embodiments, the cell transplantation is a hematopoietic cell transplantation. In some embodiments, the cell transplantation is an allogeneic bone marrow or hematopoietic stem cell transplant. In some embodiments, the compound of Formula (A) is administered concurrently with an allogeneic bone marrow or hematopoietic stem cell transplant. In some embodiments, the compound of Formula (A) is administered subsequent to an allogeneic bone marrow or hematopoietic stem cell transplant. In some embodiments, the amount of the ACK inhibitor compound (e.g., a compound of Formula (A)) prevents or reduces cGVHD while maintaining a graft-versus-leukemia (GVL) reaction effective to reduce or eliminate the number of cancerous cells in the blood of the patient. In some embodiments, the compound of Formula (A) is administered at a dosage of between about 0.1 mg/kg per day to about 100 mg/kg per day. In some embodiments, the amount of the compound of Formula (A) administered is about 40 mg/day, about 140 mg/day, about 420 mg/day, about 560 mg/day, or about 840 mg/day. In some embodiments, the compound of Formula (A) is administered from day 1 to about day 1000 following allogeneic bone marrow or hematopoietic stem cell transplant. In some embodiments, the compound of Formula (A) is administered from the onset of alloantibody driven cGVHD symptoms to about day 1000 following allogeneic bone marrow or hematopoietic stem cell transplant. In some embodiments, the compound of Formula (A) is administered orally. In some embodiments, the compound of Formula (A) is administered in combination with one or more additional therapeutic agents.

In some embodiments, disclosed herein is a method of preventing the occurrence of alloantibody driven chronic graft versus host disease (cGVHD) or reducing the severity of alloantibody driven cGVHD occurrence in a patient requiring cell transplantation, comprising administering a therapeutically effective amount of an ACK inhibitor (e.g., an ITK or BTK inhibitor). In some embodiments, disclosed herein is a method of preventing the occurrence of alloantibody driven chronic graft versus host disease (cGVHD) or reducing the severity of alloantibody driven cGVHD occurrence in a patient requiring cell transplantation, comprising administering a therapeutically effective amount of a compound of Formula (A) having the structure:

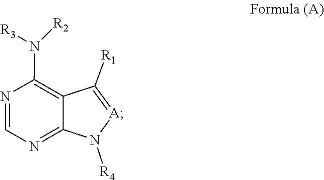

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

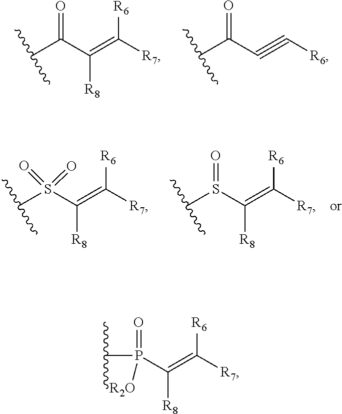

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

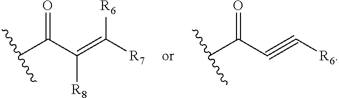

In some embodiments, disclosed herein is a method of preventing the occurrence of alloantibody driven chronic graft versus host disease (cGVHD) or reducing the severity of alloantibody driven cGVHD occurrence in a patient requiring cell transplantation, comprising administering a therapeutically effective amount of (R)-1-(3-(4-amino-3-(4-phenoxyphenyl)-1H-pyrazolo[3,4-d]pyrimidin-1-yl)piperidin-1-yl)prop-2-en-1-one (ibrutinib)

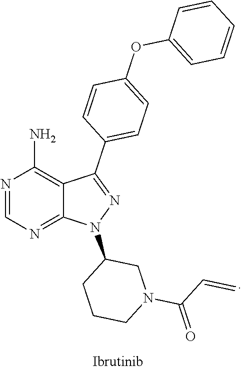 [SMILES: C=CC(=O)N1CCC[C@@H](n2nc(-c3ccc(Oc4ccccc4)cc3)c3c(N)ncnc32)C1]

In some embodiments the alloantibody driven cGVHD is non-sclerodermatous cGVHD. In some embodiments the alloantibody driven cGVHD is multi-organ cGVHD. In some embodiments the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the patient has cancer. In some embodiments, the patient has a hematologic malignancy. In some embodiments, the patient has a B-cell malignancy. In some embodiments, the patient has a T-cell malignancy. In some embodiments, the patient has a leukemia, a lymphoma, or a myeloma. In some embodiments, the amount of ibrutinib prevents or reduces alloantibody driven cGVHD while maintaining a graft-versus-leukemia (GVL) reaction effective to reduce or eliminate the number of cancerous cells in the blood of the patient. In some embodiments, the cell transplantation is a hematopoietic cell transplantation. In some embodiments, the patient has or will receive an allogeneic bone marrow or hematopoietic stem cell transplant. In some embodiments, ibrutinib is administered concurrently with an allogeneic bone marrow or hematopoietic stem cell transplant. In some embodiments, ibrutinib is administered prior to an allogeneic bone marrow or hematopoietic stem cell transplant.

Disclosed herein, in some embodiments, is a method of treating a patient for alleviation of an alloantibody response, with alleviation of consequently developed chronic graft versus host disease (cGVHD), comprising administering to the patient allogeneic hematopoietic stem cells and/or allogeneic T-cells, and a therapeutically effective amount of an ACK inhibitor (e.g., an ITK or BTK inhibitor). Disclosed herein, in some embodiments, is a method of treating a patient for alleviation of an alloantibody response, with alleviation of consequently developed chronic graft versus host disease (cGVHD), comprising administering to the patient allogeneic hematopoietic stem cells and/or allogeneic T-cells, and a therapeutically effective amount of a compound of Formula (A) having the structure:

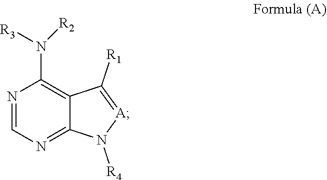

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

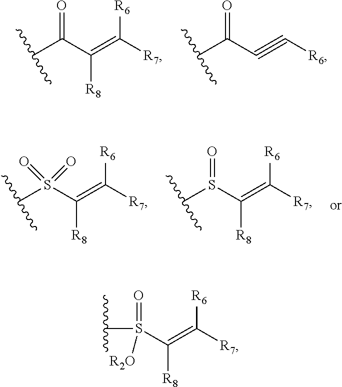

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

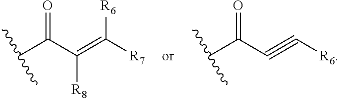

Disclosed herein, in some embodiments, is a method of treating a patient for alleviation of an alloantibody response, with alleviation of consequently developed chronic graft versus host disease (cGVHD), comprising administering to the patient allogeneic hematopoietic stem cells and/or allogeneic T-cells, and a therapeutically effective amount of (R)-1-(3-(4-amino-3-(4-phenoxyphenyl)-1H-pyrazolo[3,4-d]pyrimidin-1-yl)piperidin-1-yl)prop-2-en-1-one (ibrutinib)

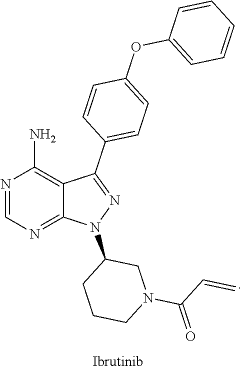 [SMILES: C=CC(=O)N1CCC[C@@H](n2nc(-c3ccc(Oc4ccccc4)cc3)c3c(N)ncnc32)C1]

In some embodiments the alloantibody driven cGVHD is non-sclerodermatous cGVHD. In some embodiments the alloantibody driven cGVHD is multi-organ cGVHD. In some embodiments the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the patient has cancer. In some embodiments, the patient as a hematologic malignancy. In some embodiments, the patient has a B-cell malignancy. In some embodiments, the patient has a T-cell malignancy. In some embodiments, the patient has a leukemia, a lymphoma, or a myeloma. In some embodiments, ibrutinib prevents or reduces alloantibody driven cGVHD while maintaining a graft-versus-leukemia (GVL) reaction effective to reduce or eliminate the number of cancerous cells in the blood of the patient. In some embodiments, the cell transplantation is a hematopoietic cell transplantation. In some embodiments, the patient has or will receive an allogeneic bone marrow or hematopoietic stem cell transplant. In some embodiments, ibrutinib is administered concurrently with an allogeneic bone marrow or hematopoietic stem cell transplant. In some embodiments, ibrutinib is administered prior to an allogeneic bone marrow or hematopoietic stem cell transplant.

In some embodiments, there are provided uses of a compound of Formula (A) for treating alloantibody driven chronic graft versus host disease (cGVHD) in a patient, wherein Formula (A) has the structure:

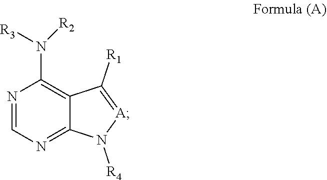

wherein:

### A is N;

R₁ is phenyl-O-phenyl or phenyl-S-phenyl;
R₂ and R₃ are independently H;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;

### G is

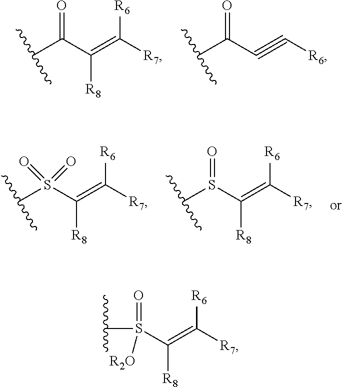

wherein,
R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or

each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

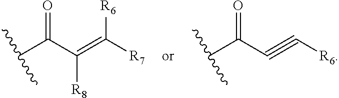

In some embodiments, the compound of Formula (A) is (R)-1-(3-(4-amino-3-(4-phenoxyphenyl)-1H-pyrazolo[3,4-d]pyrimidin-1-yl)piperidin-1-yl)prop-2-en-1-one (ibrutinib)

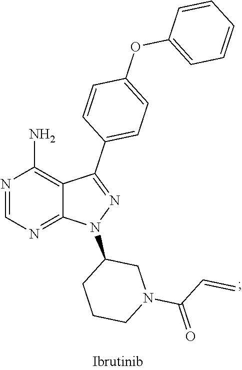 [SMILES: C=CC(=O)N1CCC[C@@H](n2nc(-c3ccc(Oc4ccccc4)cc3)c3c(N)ncnc32)C1]

or a pharmaceutically acceptable salt thereof. In some embodiments, the patient exhibits one or more symptoms of cGVHD. In some embodiments, the cGVHD is treatment naive cGVHD. In some embodiments, the cGVHD is non-sclerodermatous cGVHD. In some embodiments, the cGVHD is multi-organ cGVHD. In some embodiments, the cGVHD is bronchiolitis obliterans syndrome. In some embodiments, the cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, fibrosis is reduced. In some embodiments, lung fibrosis is reduced. In some embodiments, liver fibrosis is reduced. In some embodiments, immunoglobulin (Ig) deposition in tissue is reduced. In some embodiments, the patient has cancer. In some embodiments, the patient has a hematological malignancy. In some embodiments, the patient has a relapsed or refractory hematological malignancy. In some embodiments, the patient has a B-cell malignancy. In some embodiments, the patient has a T-cell malignancy. In some embodiments, the patient has a leukemia, a lymphoma, or a myeloma. In some embodiments, the B-cell malignancy is a non-Hodgkin's lymphoma. In some embodiments, the B-cell malignancy is chronic lymphocytic leukemia (CLL). In some embodiments, the B-cell malignancy is a relapsed or refractory B-cell malignancy. In some embodiments, the B-cell malignancy is a relapsed or refractory non-Hodgkin's lymphoma. In some embodiments, the B-cell malignancy is a relapsed or refractory CLL. In some embodiments, the patient has high risk CLL. In some embodiments, the patient has a 17p chromosomal deletion. In some embodiments, the patient has 10%, 20%, 30%, 40%, 50%, 60%, 70%, 80%, 90%, or greater CLL as determined by bone marrow biopsy. In some embodiments, the patient has received one or more prior anticancer agents. In some embodiments, the patient has received a cell transplantation. In some embodiments, the cell transplantation is a hematopoietic cell transplantation. In some embodiments, the cell transplantation is an allogeneic bone marrow or hematopoietic stem cell transplant. In some embodiments, the compound of Formula (A) is administered concurrently with an allogeneic bone marrow or hematopoietic stem cell transplant. In some embodiments, the compound of Formula (A) is administered subsequent to an allogeneic bone marrow or hematopoietic stem cell transplant. In some embodiments, the amount of the ACK inhibitor compound (e.g., a compound of Formula (A)) prevents or reduces cGVHD while maintaining a graft-versus-leukemia (GVL) reaction effective to reduce or eliminate the number of cancerous cells in the blood of the patient. In some embodiments, the compound of Formula (A) is in an amount corresponding to a dosage of between about 0.1 mg/kg per day to about 100 mg/kg per day. In some embodiments, the compound of Formula (A) is in an amount of about 40 mg/day, about 140 mg/day, about 420 mg/day, about 560 mg/day, or about 840 mg/day. In some embodiments, the compound of Formula (A) is administered from day 1 to about day 1000 following allogeneic bone marrow or hematopoietic stem cell transplant. In some embodiments, the compound of Formula (A) is administered from the onset of alloantibody driven cGVHD symptoms to about day 1000 following allogeneic bone marrow or hematopoietic stem cell transplant. In some embodiments, the compound of Formula (A) is suitable for oral administration. In some embodiments, the compound of Formula (A) is administered in combination with one or more additional therapeutic agents.

### Certain Terminology

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, “amelioration” refers to any lessening of severity, delay in onset, slowing of progression, or shortening of duration of alloantibody driven cGVHD, whether permanent or temporary, lasting or transient that can be attributed to or associated with administration of the compound or composition.

As used herein, “ACK” and “Accessible Cysteine Kinase” are synonyms. They mean a kinase with an accessible cysteine residue. ACKs include, but are not limited to, BTK, ITK, Bmx/ETK, TEC, EFGR, HER4, HER4, LCK, BLK, C-src, FGR, Fyn, HCK, Lyn, YES, ABL, Brk, CSK, FER, JAK3, SYK. In some embodiments, the ACK is a TEC family kinase. In some embodiments, the ACK is HER4. In some embodiments, the ACK is BTK. In some embodiments, the ACK is ITK.

The term “Bruton's tyrosine kinase,” as used herein, refers to Bruton's tyrosine kinase from Homo sapiens, as disclosed in, e.g., U.S. Pat. No. 6,326,469 (GenBank Accession No. NP_000052).

The term “Bruton's tyrosine kinase homolog,” as used herein, refers to orthologs of Bruton's tyrosine kinase, e.g., the orthologs from mouse (GenBank Accession No. AAB47246), dog (GenBank Accession No. XP_549139.), rat (GenBank Accession No. NP_001007799), chicken (GenBank Accession No. NP_989564), or zebra fish (GenBank Accession No. XP_698117), and fusion proteins of any of the foregoing that exhibit kinase activity towards one or more substrates of Bruton's tyrosine kinase

(e.g., a peptide substrate having the
amino acid sequence
“AVLESEEELYSSARQ” SEQ ID NO: 1)

The term “homologous cysteine,” as used herein refers to a cysteine residue found within a sequence position that is homologous to that of cysteine 481 of Bruton's tyrosine kinase, as defined herein. For example, cysteine 482 is the homologous cysteine of the rat ortholog of Bruton's tyrosine kinase; cysteine 479 is the homologous cysteine of the chicken ortholog; and cysteine 481 is the homologous cysteine in the zebra fish ortholog. In another example, the homologous cysteine of TXK, a Tec kinase family member related to Bruton's tyrosine, is Cys 350.

The term “irreversible BTK inhibitor,” as used herein, refers to an inhibitor of BTK that can form a covalent bond with an amino acid residue of BTK. In one embodiment, the irreversible inhibitor of BTK can form a covalent bond with a Cys residue of BTK; in particular embodiments, the irreversible inhibitor can form a covalent bond with a Cys 481 residue (or a homolog thereof) of BTK or a cysteine residue in the homologous corresponding position of another tyrosine kinase.

The terms “individual”, “patient” and “subject” are used interchangeable. They refer to a mammal (e.g., a human) which is the object of treatment, or observation. The term is not to be construed as requiring the supervision of a medical practitioner (e.g., a physician, physician's assistant, nurse, orderly, hospice care worker).

The terms “treat,” “treating” or “treatment”, as used herein, include lessening of severity of alloantibody driven cGVHD, delay in onset of cGVHD, causing regression of cGVHD, relieving a condition caused by of cGVHD, or stopping symptoms which result from cGVHD. The terms “treat,” “treating” or “treatment”, include, but are not limited to, prophylactic and/or therapeutic treatments.

As used herein, “alloantibody driven chronic graft versus host disease” refers to chronic GVHD that develops in part due to alloantibody production following an allogeneic transplant, such as a hematopoietic stem cell transplant. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD.

### Graft Versus Host Disease

Described herein, in some embodiments, are methods of treating alloantibody driven chronic graft versus host disease (cGVHD) in a patient in need thereof comprising administering to the patient a composition comprising a therapeutically-effective amount of an ACK inhibitor compound (e.g., an ITK or BTK inhibitor, such as, ibrutinib), thereby treating the alloantibody driven cGVHD. In some embodiments, the alloantibody driven cGVHD is treatment naive cGVHD. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the patient has received a hematopoietic cell transplantation. In some embodiments, the patient has received a peripheral blood stem cell transplantation. In some embodiments, the patient has received a bone marrow transplantation. In some embodiments, the ACK inhibitor compound (e.g., an ITK or BTK inhibitor, such as, ibrutinib) is administered prior to administration of the cell transplant. In some embodiments, the ACK inhibitor compound (e.g., an ITK or BTK inhibitor, such as, ibrutinib) is administered subsequent to administration of the cell transplant. In some embodiments, the ACK inhibitor compound (e.g., an ITK or BTK inhibitor, such as, ibrutinib) is administered concurrently with administration of the cell transplant. In some embodiments, the ACK inhibitor compound (e.g., an ITK or BTK inhibitor, such as, ibrutinib) is administered after the onset of symptoms of alloantibody driven cGVHD. In some embodiments, the patient exhibits one or more symptoms of alloantibody driven cGVHD.

Further described herein, in some embodiments, are methods of preventing the occurrence of alloantibody driven chronic graft versus host disease (cGVHD) or reducing the severity of alloantibody driven cGVHD occurrence in a patient requiring cell transplantation comprising administering to the patient a composition comprising a therapeutically-effective amount of an ACK inhibitor compound (e.g., an ITK or BTK inhibitor, such as, ibrutinib). In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the patient requires hematopoietic cell transplantation. In some embodiments, the patient requires peripheral blood stem cell transplantation. In some embodiments, the patient requires bone marrow transplantation. In some embodiments, the ACK inhibitor compound (e.g., an ITK or BTK inhibitor, such as, ibrutinib) is administered prior to administration of the cell transplant. In some embodiments, the ACK inhibitor compound (e.g., an ITK or BTK inhibitor, such as, ibrutinib) is administered subsequent to administration of the cell transplant. In some embodiments, the ACK inhibitor compound (e.g., an ITK or BTK inhibitor, such as, ibrutinib) is administered concurrently with administration of the cell transplant. In some embodiments, the patient exhibits one or more symptoms of alloantibody driven cGVHD.

Described herein, in some embodiments, are methods of treating alloantibody driven chronic graft versus host disease (cGVHD) in a patient in need thereof comprising administering to the patient a composition comprising a therapeutically-effective amount of ibrutinib, thereby treating the alloantibody driven cGVHD. In some embodiments, the alloantibody driven cGVHD is treatment naive cGVHD. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the patient has received a hematopoietic cell transplantation. In some embodiments, the patient has received a peripheral blood stem cell transplantation. In some embodiments, the patient has received bone marrow transplantation. In some embodiments, the ibrutinib is administered prior to administration of the cell transplant. In some embodiments, the ibrutinib is administered subsequent to administration of the cell transplant. In some embodiments, the ibrutinib is administered concurrently with administration of the cell transplant. In some embodiments, the ibrutinib is administered after the onset of symptoms of alloantibody driven cGVHD. In some embodiments, the patient exhibits one or more symptoms of alloantibody driven cGVHD.

Described herein are methods of preventing the occurrence of alloantibody driven chronic graft versus host disease (cGVHD) or reducing the severity of alloantibody driven cGVHD occurrence in a patient requiring stem cell transplantation comprising administering to the patient a composition comprising a therapeutically-effective amount of ibrutinib. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the patient requires hematopoietic stem cell transplantation. In some embodiments, the patient requires peripheral blood stem cell transplantation. In some embodiments, the patient requires bone marrow transplantation. In some embodiments, ibrutinib is administered prior to administration of the stem cell transplant. In some embodiments, ibrutinib is administered subsequent to administration of the stem cell transplant. In some embodiments, ibrutinib is administered concurrently with administration of the stem cell transplant. In some embodiments, ibrutinib is administered prior to, subsequent to, or concurrently with administration of allogeneic hematopoietic stem cells and/or allogeneic T-cells.

Further described herein are methods of treating a patient for alleviation of an alloantibody response, with alleviation of consequently developed chronic graft versus host disease (cGVHD), comprising administering to the patient allogeneic hematopoietic stem cells and/or allogeneic T-cells, wherein a therapeutically effective amount of an ACK inhibitor compound (e.g., a BTK inhibitor, such as for example ibrutinib) is administered prior to, subsequently, or concurrently with administration of the allogeneic hematopoietic stem cells and/or allogeneic T-cells.

Treatment of proliferative blood disorders, such as leukemia, lymphoma and myeloma usually involves one or more forms of chemotherapy and/or radiation therapy. These treatments destroy malignant cells, but also destroy healthy blood cells. Allogeneic hematopoietic cell transplantation is an effective therapy for the treatment of many hematologic malignancies, including, for example, B-cell and T-cell malignancies. In allogeneic hematopoietic cell transplantation, bone marrow (or, in some cases, peripheral blood) from an unrelated or a related (but not identical twin) donor is used to replace the healthy blood cells destroyed in the cancer patient. The bone marrow (or peripheral blood) contains stem cells, which are the precursors to all the different cell types (e.g., red cells, phagocytes, platelets and lymphocytes) found in blood. Allogeneic hematopoietic cell transplantation is known to have both a restorative effect and a curative effect. The restorative effect arises from the ability of the stem cells to repopulate the cellular components of blood. The curative properties of allogeneic hematopoietic cell transplantation derive largely from a graft-versus-leukemia (GVL) effect. The transplanted hematopoietic cells from the donor (specifically, the T lymphocytes) attack the cancerous cells, enhancing the suppressive effects of the other forms of treatment. Essentially, the GVL effect comprises an attack on the cancerous cells by the blood cells derived from the transplantation, making it less likely that the malignancy will return after transplant. Controlling the GVL effect prevents escalation of the GVL effect into GVHD. A similar effect against tumors (graft-versus tumor) is also known.

Allogeneic hematopoietic cell transplantation is often toxic to the patient. This toxicity arises from the difficulty in dissociating the GVL or GVT effect from graft-versus-host disease (GVHD), an often-lethal complication of allogeneic BMT.

GVHD is a major complication of allogeneic hematopoietic cell transplant (HCT). GVHD is an inflammatory disease initiated by T cells in the donor graft that recognize histocompatibility and other tissue antigens of the host and GVHD is mediated by a variety of effector cells and inflammatory cytokines. GVHD presents in both acute and chronic forms. The most common symptomatic organs are the skin, liver, and gastrointestinal tract. GVHD may involve other organs such as the lung. Treatment of GVHD is generally only 50-75% successful; the remainder of patients generally do not survive. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of HLA-matched unrelated donor marrow, and in a higher percentage of recipient of HLA-mismatched marrow. Patients with mild intestinal GVHD present with anorexia, nausea, vomiting, abdominal pain and diarrhea, whereas patients with severe GVHD are disabled by these symptoms. If untreated, symptoms of intestinal GVHD persist and often progress; spontaneous remissions are unusual. In its most severe form, GVHD leads to necrosis and exfoliation of most of the epithelial cells of the intestinal mucosa, a frequently fatal condition. The symptoms of acute GVHD usually present within 100 days of transplantation. The symptoms of chronic GVHD usually present somewhat later, up to three years after allogeneic HCT, and are often proceeded by a history of acute GVHD.

Described herein are methods of preventing the occurrence of alloantibody driven chronic graft versus host disease (cGVHD) or reducing the severity of alloantibody driven cGVHD occurrence in a patient requiring cell transplantation comprising administering to the patient a composition comprising a therapeutically-effective amount ibrutinib. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the patient requires hematopoietic cell transplantation. Further described herein are methods of treating a patient for alleviation of a bone marrow mediated disease, with alleviation of consequently developed graft versus host disease (GVHD), comprising administering to the patient allogeneic hematopoietic stem cells and/or allogeneic T-cells, wherein a therapeutically effective amount of ibrutinib is administered prior to or concurrently with the allogeneic hematopoietic stem cells and/or allogeneic T-cells. In some embodiments, the patient has cancer. In some embodiments, the patient has a hematologic malignancy. In some embodiments, the patient has a B-cell malignancy. In some embodiments, the patient has a T-cell malignancy. In some embodiments, the patient has a leukemia, lymphoma, or a myeloma. In some embodiments, a compound disclosed herein prevents or reduces cGVHD while maintaining a graft-versus-leukemia (GVL) reaction effective to reduce or eliminate the number of cancerous cells in the blood of the patient. In some embodiments, the patient has or will receive an allogeneic bone marrow or hematopoietic stem cell transplant. In some embodiments, ibrutinib is administered concurrently with an allogeneic bone marrow or hematopoietic stem cell transplant. In some embodiments, ibrutinib is administered prior to an allogeneic bone marrow or hematopoietic stem cell transplant. In some embodiments, ibrutinib is administered subsequent to an allogeneic bone marrow or hematopoietic stem cell transplant.

In some embodiments, the patient has a non-Hodgkin lymphoma. In some embodiments, the patient has a Hodgkin lymphoma. In some embodiments, the patient has a B-cell malignancy.

Disclosed herein, in some embodiments, are methods of treating a patient for alleviation of an alloantibody response, with alleviation of consequently developed chronic graft versus host disease (cGVHD), comprising administering to the patient allogeneic hematopoietic stem cells and/or allogeneic T-cells, with a therapeutically effective amount of a BTK inhibitor.

Disclosed herein, in some embodiments, are methods of treating alloantibody driven chronic graft versus host disease (cGVHD) in a patient in need thereof comprising administering to the patient a composition comprising a therapeutically-effective amount of a BTK inhibitor, thereby treating the alloantibody driven cGVHD. In some embodiments, the alloantibody driven cGVHD is treatment naive cGVHD. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, fibrosis is reduced. In some embodiments, lung fibrosis is reduced. In some embodiments, liver fibrosis is reduced. In some embodiments, immunoglobulin (Ig) deposition in tissue is reduced. In some embodiments, the patient has received a hematopoietic cell transplantation. In some embodiments, the patient has received a peripheral blood stem cell transplantation. In some embodiments, the patient has received a bone marrow transplantation. In some embodiments, the BTK inhibitor is administered prior to administration of the cell transplant. In some embodiments, the BTK inhibitor is administered subsequent to administration of the cell transplant. In some embodiments, the BTK inhibitor is administered concurrently with administration of the cell transplant. In some embodiments, the BTK inhibitor is administered after the onset of symptoms of alloantibody driven cGVHD. In some embodiments, the patient exhibits one or more symptoms of alloantibody driven cGVHD.

In some embodiments, described herein, are methods of preventing the occurrence of alloantibody driven chronic graft versus host disease (cGVHD) or reducing the severity of alloantibody driven cGVHD occurrence in a patient requiring cell transplantation comprising administering to the patient a composition comprising a therapeutically-effective amount of a BTK inhibitor. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the patient requires hematopoietic cell transplantation. In some embodiments, the patient requires peripheral blood stem cell transplantation. In some embodiments, the patient requires bone marrow transplantation. In some embodiments, the BTK inhibitor is administered prior to administration of the cell transplant. In some embodiments, the BTK inhibitor is administered subsequent to administration of the cell transplant. In some embodiments, the BTK inhibitor is administered concurrently with administration of the cell transplant. In some embodiments, the BTK inhibitor is administered prior to, subsequent to, or concurrently with administration of allogeneic hematopoietic stem cells and/or allogeneic T-cells. In some embodiments, the patient exhibits one or more symptoms of alloantibody driven cGVHD.

Disclosed herein, in some embodiments, is a method of treating a patient for alleviation of an alloantibody response, with alleviation of consequently developed chronic graft versus host disease (cGVHD), comprising administering to the patient allogeneic hematopoietic stem cells and/or allogeneic T-cells, with a therapeutically effective amount of an ITK inhibitor.

Disclosed herein, in some embodiments, are methods of treating alloantibody driven chronic graft versus host disease (cGVHD) in a patient in need thereof comprising administering to the patient a composition comprising a therapeutically-effective amount of a ITK inhibitor, thereby treating the alloantibody driven cGVHD. In some embodiments, the alloantibody driven cGVHD is treatment naive cGVHD. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the patient has received a hematopoietic cell transplantation. In some embodiments, the patient has received a peripheral blood stem cell transplantation. In some embodiments, the patient has received a bone marrow transplantation. In some embodiments, the ITK inhibitor is administered prior to administration of the cell transplant. In some embodiments, the ITK inhibitor is administered subsequent to administration of the cell transplant. In some embodiments, the ITK inhibitor is administered concurrently with administration of the cell transplant. In some embodiments, the ITK inhibitor is administered after the onset of symptoms of alloantibody driven cGVHD. In some embodiments, the patient exhibits one or more symptoms of alloantibody driven cGVHD.

In some embodiments, described herein, are methods of preventing the occurrence of alloantibody driven chronic graft versus host disease (cGVHD) or reducing the severity of alloantibody driven cGVHD occurrence in a patient requiring cell transplantation comprising administering to the patient a composition comprising a therapeutically-effective amount of an ITK inhibitor. In some embodiments, the alloantibody driven cGVHD is non-sclerodermatous cGVHD. In some embodiments, the alloantibody driven cGVHD is multi-organ cGVHD. In some embodiments, the alloantibody driven cGVHD is bronchiolitis obliterans syndrome. In some embodiments, the alloantibody driven cGVHD is lung cGVHD. In some embodiments, the patient requires hematopoietic cell transplantation. In some embodiments, the patient requires peripheral blood stem cell transplantation. In some embodiments, the patient requires bone marrow transplantation. In some embodiments, the ITK inhibitor is administered prior to administration of the cell transplant. In some embodiments, the ITK inhibitor is administered subsequent to administration of the cell transplant. In some embodiments, the ITK inhibitor is administered concurrently with administration of the cell transplant. In some embodiments, the ITK inhibitor is administered prior to, subsequent to, or concurrently with administration of allogeneic hematopoietic stem cells and/or allogeneic T-cells. In some embodiments, the patient exhibits one or more symptoms of alloantibody driven cGVHD.

### Combination Therapies

Described herein, in some embodiments, are methods of treating alloantibody driven chronic graft versus host disease (cGVHD) in a patient in need thereof, comprising administering a therapeutically effective amount of an ACK inhibitor (e.g., an ITK or BTK inhibitor) and an additional therapeutic agent.

Further described herein are methods of preventing the occurrence of alloantibody driven chronic graft versus host disease (cGVHD) or reducing the severity of alloantibody driven cGVHD occurrence in a patient requiring cell transplantation comprising administering to the patient a composition comprising a therapeutically-effective amount of an ACK inhibitor compound (e.g., an ITK or BTK inhibitor, such as, for example, ibrutinib) and an additional therapeutic agent.

Further described herein, in some embodiments, are methods of treating a patient for alleviation of a, with alleviation of consequently developed chronic graft versus host disease (cGVHD), comprising administering to the patient allogeneic hematopoietic stem cells and/or allogeneic T-cells, wherein a therapeutically effective amount of an ACK inhibitor compound (e.g., an ITK or BTK inhibitor, such as, for example, ibrutinib) and an additional therapeutic agent is administered prior to or concurrently with the allogeneic hematopoietic stem cells and/or allogeneic T-cells. In some embodiments, the individual is administered an additional therapy such as, but not limited to, extracorporeal photopheresis or infusion of mesenchymal stem cells or donor lymphocytes.

In some embodiments, the additional therapeutic agent is an anti-GVHD therapeutic agent. In some embodiments, the anti-GVHD therapeutic agent is an immunosuppressive drug. In some embodiments, the immunosuppressive drug includes cyclosporine, tacrolimus, methotrexate, mycophenolate mofetil, corticosteroids, azathioprine or antithymocyte globulin (ATG). In some embodiments, the immunosuppressive drug is a monoclonal antibody (for example, anti-CD3, anti-CD5, and anti-IL-2 antibodies). In some embodiments, the immunosuppressive drug is Mycophenolate mofetil, Alemtuzumab, Antithymocyte globulin (ATG), Sirolimus, Tacrolimus, Thalidomide, Daclizumab, Infliximab, or Clofazimine are of use to treat chronic GVHD. In some embodiments, the additional therapeutic agent is denileukin diftitox, defibrotide, budesonide, beclomethasone dipropionate, or pentostatin.

In some embodiments, the additional therapeutic agent is an IL-6 receptor inhibitor. In some embodiments, the additional therapeutic agent is an IL-6 receptor antibody.

In some embodiments, the additional therapeutic agent is a TLR5 agonist.

In some embodiments, the patient undergoes an additional therapy such as extracorporeal photopheresis or infusion of mesenchymal stem cells or donor lymphocytes.

In some embodiments, the additional therapeutic agent is a topically active corticosteroid (TAC). In some embodiments, the TAC is beclomethasone dipropionate, alciometasone dipropionate, busedonide, 22S busesonide, 22R budesonide, beclomethasone-17-monopropionate, betamethasone, clobetasol propionate, dexamethasone, diflorasone diacetate, flunisolide, fluocinonide, flurandrenolide, fluticasone propionate, halobetasol propionate, halcinocide, mometasone furoate, triamcinalone acetonide or a combination thereof.

In some embodiments, the additional therapeutic agent is an antifungal agent. In some embodiments, the additional therapeutic agent is nystatin, clotrimazole, amphotericin, fluconazole itraconazole or a combination thereof.

In some embodiments, the additional therapeutic agent is a sialogogue. In some embodiments, the additional therapeutic agent is cevimeline, pilocarpine, bethanechol or a combination thereof.

In some embodiments, the additional therapeutic agent is a topical anesthetic. In some embodiments, the additional therapeutic agent is lidocaine, dyclonine, diphenhydramine, doxepin or a combination thereof.

In the methods described herein, any suitable technique for chemotherapy, biotherapy, immunosuppression and radiotherapy known in the art may be used. For example, the chemotherapeutic agent may be any agent that exhibits an oncolytic effect against cancer cells or neoplastic cells of the subject. For example, the chemotherapeutic agent may be, without limitation, an anthracycline, an alkylating agent, an alkyl sulfonate, an aziridine, an ethylenimine, a methyhnelamine, a nitrogen mustard, a nitrosourea, an antibiotic, an antimetabolite, a folic acid analogue, a purine analogue, a pyrimidine analogue, an enzyme, a podophyllotoxin, a platinum-containing agent or a cytokine. Preferably, the chemotherapeutic agent is one that is known to be effective against the particular cell type that is cancerous or neoplastic. In some embodiments, the chemotherapeutic agent is effective in the treatment of hematopoietic malignancies, such as thiotepa, cisplatin-based compounds, and cyclophosphamide. Cytokines include interferons, G-CSF, erythropoietin, GM-CSF, interleukins, parathyroid hormone, and the like. Biotherapies include alemtuzumab, rituximab, bevacizumab, vascular disrupting agents, lenalidomide, and the like. Radiosensitizers include nicotinomide, and the like.

In some embodiments, the ACK inhibitor is administered in combination with a chemotherapeutic agent or biologic agent selected from among an antibody, a B cell receptor pathway inhibitor, a T cell receptor inhibitor, a PI3K inhibitor, an IAP inhibitor, an mTOR inhibitor, a radioimmunotherapeutic, a DNA damaging agent, a histone deacetylase inhibitor, a protein kinase inhibitor, a hedgehog inhibitor, an Hsp90 inhibitor, a telomerase inhibitor, a Jak1/2 inhibitor, a protease inhibitor, an IRAK inhibitor, a PKC inhibitor, a PARP inhibitor, a CYP3A4 inhibitor, an AKT inhibitor, an Erk inhibitor, a proteosome inhibitor, an alkylating agent, an anti-metabolite, a plant alkaloid, a terpenoid, a cytotoxin, a topoisomerase inhibitor, or a combination thereof. In some embodiments, the B cell receptor pathway inhibitor is a CD79A inhibitor, a CD79B inhibitor, a CD19 inhibitor, a Lyn inhibitor, a Syk inhibitor, a PI3K inhibitor, a Blnk inhibitor, a PLCγ inhibitor, a PKCβ inhibitor, a CD22 inhibitor, a Bcl-2 inhibitor, an IRAK 1/4 inhibitor, a JAK inhibitor (e.g., ruxolitinib, baricitinib, CYT387, lestauritinib, pacritinib, TG101348, SAR302503, tofacitinib (Xeljanz), etanercept (Enbrel), GLPG0634, R256), a microtubule inhibitor, a Topo II inhibitor, anti-TWEAK antibody, anti-IL17 bispecific antibody, a CK2 inhibitor, anaplastic lymphoma kinase (ALK) and c-Met inhibitors, demethylase enzyme inhibitors such as demethylase, HDM, LSDI and KDM, fatty acid synthase inhibitors such as spirocyclic piperidine derivatives, glucocorticosteriod receptor agonist, fusion anti-CD 19-cytotoxic agent conjugate, antimetabolite, p70S6K inhibitor, immune modulators, AKT/PKB inhibitor, procaspase-3 activator PAC-1, BRAF inhibitor, lactate dehydrogenase A (LDH-A) inhibitor, CCR2 inhibitor, CXCR4 inhibitor, chemokine receptor antagonists, DNA double stranded break repair inhibitors, NOR202, GA-101, TLR2 inhibitor, or a combination thereof. In some embodiments, the T cell receptor inhibitor is Muromonab-CD3. In some embodiments, the chemotherapeutic agent is selected from among rituximab (rituxan), carfilzomib, fludarabine, cyclophosphamide, vincristine, prednisalone. chlorambucil, ifosphamide, doxorubicin, mesalazine, thalidomide, revlimid, lenalidomide, temsirolimus, everolimus, fostamatinib, paclitaxel, docetaxel, ofatumumab, dexamethasone, bendamustine, prednisone, CAL-101, ibritumomab, tositumomab, bortezomib, pentostatin, endostatin, ritonavir, ketoconazole, an anti-VEGF antibody, herceptin, cetuximab, cisplatin, carboplatin, docetaxel, erlotinib, etopiside, 5-fluorouracil, gemcitabine, ifosphamide, imatinib mesylate (Gleevec), gefitinib, erlotinib, procarbazine, prednisone, irinotecan, leucovorin, mechlorethamine, methotrexate, oxaliplatin, paclitaxel, sorafenib, sunitinib, topotecan, vinblastine, GA-1101, dasatinib, Sipuleucel-T, disulfiram, epigallocatechin-3-gallate, salinosporamide A, ONX0912, CEP-18770, MLN9708, R-406, lenalinomide, spirocyclic piperidine derivatives, quinazoline carboxamide azetidine compounds, thiotepa, DWA2114R, NK121, IS 3 295, 254-S, alkyl sulfonates such as busulfan, improsulfan and piposulfan; aziridines such as benzodepa, carboquone, meturedepa and uredepa; ethylenimine, methylmelamines such as altretamine, triethylenemelamine, triethylenephosphoramide, triethylenethiophosphoramide and trimethylmelamine; chlornaphazine; estramustine; ifosfamide; mechlorethamine; oxide hydrochloride; novobiocin; phenesterine; prednimustine; trofosfamide; uracil mustard; nitrosoureas such as carmustine, chlorozotocin, fotemustine, lomustine, nimustine, ranimustine; antibiotics such as aclacinomycins, actinomycin, anthramycin, azaserine, bleomycins, cactinomycin, calicheamicin, carubicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatrexate; defosfamide; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; taxoids, e.g., paclitaxel and docetaxel; 6-thioguanine; mercaptopurine; methotrexate; platinum analogs; platinum; etoposide (VP-16); ifosfamide; mitomycin C; mitoxantrone; vincristine; vinorelbine; Navelbine; Novantrone; teniposide; daunomycin; aminopterin; Xeloda; ibandronate; CPT1 1; topoisomerase inhibitor RFS 2000; difluoromethylornithine (DMFO); retinoic acid; esperamycins; capecitabine; and pharmaceutically acceptable salts, acids or derivatives of, anti-hormonal agents such as anti-estrogens including for example tamoxifen, raloxifene, aromatase inhibiting 4(5)-imidazoles, 4-hydroxytamoxifen, trioxifene, keoxifene, LY117018, onapristone and toremifene (Fareston); antiandrogens such as flutamide, nilutamide, bicalutamide, leuprolide and goserelin; ACK inhibitors such as AVL-263 (Avila Therapeutics/Celgene Corporation), AVL-292 (Avila Therapeutics/Celgene Corporation), AVL-291 (Avila Therapeutics/Celgene Corporation), BMS-488516 (Bristol-Myers Squibb), BMS-509744 (Bristol-Myers Squibb), CGI-1746 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or a combination thereof.

When an additional agent is co-administered with an ACK inhibitor, the additional agent and the ACK inhibitor do not have to be administered in the same pharmaceutical composition, and are optionally, because of different physical and chemical characteristics, administered by different routes. The initial administration is made, for example, according to established protocols, and then, based upon the observed effects, the dosage, modes of administration and times of administration are modified.

By way of example only, if a side effect experienced by an individual upon receiving an ACK inhibitor is nausea, then it is appropriate to administer an anti-emetic agent in combination with the ACK inhibitor.

Or, by way of example only, the therapeutic effectiveness of an ACK inhibitor described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by an individual is increased by administering an ACK inhibitor described herein with another therapeutic agent (which also includes a therapeutic regimen) that also has therapeutic benefit. In any case, regardless of the disease, disorder being treated, the overall benefit experienced by the patient is in some embodiments simply additive of the two therapeutic agents or in other embodiments, the patient experiences a synergistic benefit.

The particular choice of compounds used will depend upon the diagnosis of the attending physicians and their judgment of the condition of the patient and the appropriate treatment protocol. The compounds are optionally administered concurrently (e.g., simultaneously, essentially simultaneously or within the same treatment protocol) or sequentially, depending upon the nature of the disorder, the condition of the patient, and the actual choice of compounds used. The determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature combination treatment further includes periodic treatments that start and stop at various times to assist with the clinical management of the patient.

For combination therapies described herein, dosages of the co-administered compounds will of course vary depending on the type of co-drug employed, on the specific drug employed, on the disorder being treated and so forth. In addition, when co-administered with an additional therapeutic agent, an ACK inhibitor described herein is administered either simultaneously with the additional therapeutic agent, or sequentially. If administered sequentially, the attending physician will decide on the appropriate sequence of administering protein in combination with the biologically active agent(s).

If the additional therapeutic agent and the ACK inhibitor are administered simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses is from about more than zero weeks to less than about four weeks. In addition, the combination methods, compositions and formulations are not to be limited to the use of only two agents; the use of multiple therapeutic combinations is also envisioned.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

In some embodiments, the pharmaceutical agents which make up the combination therapy disclosed herein are administered in a combined dosage form, or in separate dosage forms intended for substantially simultaneous administration. In some embodiments, the pharmaceutical agents that make up the combination therapy are administered sequentially, with either therapeutic compound being administered by a regimen calling for two-step administration. In some embodiments, the two-step administration regimen calls for sequential administration of the active agents or spaced-apart administration of the separate active agents. The time period between the multiple administration steps ranges from a few minutes to several hours, depending upon the properties of each pharmaceutical agent, such as potency, solubility, bioavailability, plasma half-life and kinetic profile of the pharmaceutical agent. In some embodiments, circadian variation of the target molecule concentration determines the optimal dose interval.

In some embodiments, the ACK inhibitor compound and the additional therapeutic agent are administered in a unified dosage form. In some embodiments, the ACK inhibitor compound and the additional therapeutic agent are administered in separate dosage forms. In some embodiments, the ACK inhibitor compound and the additional therapeutic agent are administered simultaneously or sequentially.

### Administration

Described herein, in some embodiments, are methods of treating alloantibody driven chronic graft versus host disease (cGVHD) in a patient in need thereof, comprising administering a therapeutically effective amount of an ACK inhibitor (e.g., an ITK or BTK inhibitor).

Further described herein are methods of preventing the occurrence of alloantibody driven chronic graft versus host disease (cGVHD) or reducing the severity of alloantibody driven cGVHD occurrence in a patient requiring cell transplantation comprising administering to the patient a composition comprising a therapeutically-effective amount of an ACK inhibitor compound (e.g., an ITK or BTK inhibitor, such as, for example, ibrutinib).

Further described herein, in some embodiments, are methods of treating a patient for alleviation of a, with alleviation of consequently developed chronic graft versus host disease (cGVHD), comprising administering to the patient allogeneic hematopoietic stem cells and/or allogeneic T-cells, wherein a therapeutically effective amount of an ACK inhibitor compound (e.g., an ITK or BTK inhibitor, such as, for example, ibrutinib) is administered prior to or concurrently with the allogeneic hematopoietic stem cells and/or allogeneic T-cells.

The ACK inhibitor compound (e.g., an ITK or BTK inhibitor, such as for example ibrutinib) is administered before, during or after the development of cGVHD. In some embodiments, the ACK inhibitor compound (e.g., an ITK or BTK inhibitor, such as for example ibrutinib) is used as a prophylactic and is administered continuously to subjects with a propensity to develop cGVHD (e.g., allogeneic transplant recipients). In some embodiments, the ACK inhibitor compound (e.g., an ITK or BTK inhibitor, such as for example ibrutinib) is administered to an individual during or as soon as possible after the development of alloantibody driven cGVHD. In some embodiments, the administration of the ACK inhibitor compound (e.g., an ITK or BTK inhibitor, such as for example ibrutinib) is initiated within the first 48 hours of the onset of the symptoms, within the first 6 hours of the onset of the symptoms, or within 3 hours of the onset of the symptoms. In some embodiments, the initial administration of the ACK inhibitor compound (e.g., an ITK or BTK inhibitor, such as for example ibrutinib) is via any route practical, such as, for example, an intravenous injection, a bolus injection, infusion over 5 minutes to about 5 hours, a pill, a capsule, a tablet, a transdermal patch, buccal delivery, and the like, or combination thereof. The ACK inhibitor compound (e.g., an ITK or BTK inhibitor, such as for example ibrutinib) should be administered as soon as is practicable after the onset of a disorder is detected or suspected, and for a length of time necessary for the treatment of the disease, such as, for example, from about 1 month to about 3 months. The length of treatment can vary for each subject, and the length can be determined using the known criteria. In some embodiments, the ACK inhibitor compound (e.g., an ITK or BTK inhibitor, such as for example ibrutinib) is administered for at least 2 weeks, between about 1 month to about 5 years, or from about 1 month to about 3 years.

Therapeutically effective amounts will depend on the severity and course of the disorder, previous therapy, the patient's health status, weight, and response to the drugs, and the judgment of the treating physician. Prophylactically effective amounts depend on the patient's state of health, weight, the severity and course of the disease, previous therapy, response to the drugs, and the judgment of the treating physician.

In some embodiments, the ACK inhibitor compound (e.g., an ITK or BTK inhibitor, such as for example ibrutinib) is administered to the patient on a regular basis, e.g., three times a day, two times a day, once a day, every other day or every 3 days. In other embodiments, the ACK inhibitor compound (e.g., an ITK or BTK inhibitor, such as for example ibrutinib) is administered to the patient on an intermittent basis, e.g., twice a day followed by once a day followed by three times a day; or the first two days of every week; or the first, second and third day of a week. In some embodiments, intermittent dosing is as effective as regular dosing. In further or alternative embodiments, the ACK inhibitor compound (e.g., an ITK or BTK inhibitor, such as for example ibrutinib) is administered only when the patient exhibits a particular symptom, e.g., the onset of pain, or the onset of a fever, or the onset of an inflammation, or the onset of a skin disorder. Dosing schedules of each compound may depend on the other or may be independent of the other.

In the case wherein the patient's condition does not improve, upon the doctor's discretion the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance regimen is administered if necessary. Subsequently, the dosage or the frequency of administration, or both, of the ACK inhibitor compound (e.g., an ITK or BTK inhibitor, such as for example ibrutinib) can be reduced, as a function of the symptoms, to a level at which the individual's improved condition is retained. Individuals can, however, require intermittent treatment on a long-term basis upon any recurrence of symptoms.

The amount of the ACK inhibitor compound (e.g., an ITK or BTK inhibitor, such as for example ibrutinib) will vary depending upon factors such as the particular compound, disorder and its severity, the identity (e.g., weight) of the subject or host in need of treatment, and is determined according to the particular circumstances surrounding the case, including, e.g., the specific agents being administered, the routes of administration, and the subject or host being treated. In general, however, doses employed for adult human treatment will typically be in the range of 0.02-5000 mg per day, or from about 1-1500 mg per day. The desired dose may be presented in a single dose or as divided doses administered simultaneously (or over a short period of time) or at appropriate intervals, for example as two, three, four or more sub-doses per day.

In some embodiments, the therapeutic amount of the ACK inhibitor (e.g., an ITK or BTK inhibitor, such as for example ibrutinib) is from 100 mg/day up to, and including, 2000 mg/day. In some embodiments, the amount of the ACK inhibitor (e.g., an ITK or BTK inhibitor, such as for example ibrutinib) is from 140 mg/day up to, and including, 840 mg/day. In some embodiments, the amount of the ACK inhibitor (e.g., an ITK or BTK inhibitor, such as for example ibrutinib) is from 420 mg/day up to, and including, 840 mg/day. In some embodiments, the amount of the ACK inhibitor (e.g., an ITK or BTK inhibitor, such as for example ibrutinib) is about 40 mg/day. In some embodiments, the amount of the ACK inhibitor (e.g., an ITK or BTK inhibitor, such as for example ibrutinib) is about 140 mg/day. In some embodiments, the amount of the ACK inhibitor (e.g., an ITK or BTK inhibitor, such as for example ibrutinib) is about 280 mg/day. In some embodiments, the amount of the ACK inhibitor (e.g., an ITK or BTK inhibitor, such as for example ibrutinib) is about 420 mg/day. In some embodiments, the amount of the ACK inhibitor (e.g., an ITK or BTK inhibitor, such as for example ibrutinib) is about 560 mg/day. In some embodiments, the amount of the ACK inhibitor (e.g., an ITK or BTK inhibitor, such as for example ibrutinib) is about 700 mg/day. In some embodiments, the amount of the ACK inhibitor (e.g., an ITK or BTK inhibitor, such as for example ibrutinib) is about 840 mg/day. In some embodiments, the amount of the ACK inhibitor (e.g., an ITK or BTK inhibitor, such as for example ibrutinib) is about 980 mg/day. In some embodiments, the amount of the ACK inhibitor (e.g., an ITK or BTK inhibitor, such as for example ibrutinib) is about 1120 mg/day. In some embodiments, the amount of the ACK inhibitor (e.g., an ITK or BTK inhibitor, such as for example ibrutinib) is about 1260 mg/day. In some embodiments, the amount of the ACK inhibitor (e.g., an ITK or BTK inhibitor, such as for example ibrutinib) is about 1400 mg/day. In some embodiments, a compound of Formula (A) is administered at a dosage of between about 0.1 mg/kg per day to about 100 mg/kg per day.

In some embodiments, the dosage of the ACK inhibitor (e.g., an ITK or BTK inhibitor, such as for example ibrutinib) is escalated over time. In some embodiments, the dosage of the ACK inhibitor (e.g., an ITK or BTK inhibitor, such as for example ibrutinib) is escalated, for example, from at or about 1.25 mg/kg/day to at or about 12.5 mg/kg/day over a predetermined period of time. In some embodiments the predetermined period of time is over 1 month, over 2 months, over 3 months, over 4 months, over 5 months, over 6 months, over 7 months, over 8 months, over 9 months, over 10 months, over 11 months, over 12 months, over 18 months, over 24 months or longer.

The ACK inhibitor compound (e.g., an ITK or BTK inhibitor, such as for example ibrutinib) may be formulated into unit dosage forms suitable for single administration of precise dosages. In unit dosage form, the formulation is divided into unit doses containing appropriate quantities of one or both compounds. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

It is understood that a medical professional will determine the dosage regimen in accordance with a variety of factors. These factors include the severity of GVHD in the subject, as well as the age, weight, sex, diet, and medical condition of the subject.

### Compounds

Described herein, in some embodiments, are methods of treating alloantibody driven chronic graft versus host disease (cGVHD) in a patient in need thereof, comprising administering a therapeutically effective amount of an ACK inhibitor (e.g., an ITK or BTK inhibitor).

Further described herein are methods of preventing the occurrence of alloantibody driven chronic graft versus host disease (cGVHD) or reducing the severity of alloantibody driven cGVHD occurrence in a patient requiring cell transplantation comprising administering to the patient a composition comprising a therapeutically-effective amount of an ACK inhibitor compound (e.g., an ITK or BTK inhibitor, such as, for example, ibrutinib).

Further described herein, in some embodiments, are methods of treating a patient for alleviation of an alloantibody response, with alleviation of consequently developed chronic graft versus host disease (cGVHD), comprising administering to the patient allogeneic hematopoietic stem cells and/or allogeneic T-cells, wherein a therapeutically effective amount of an ACK inhibitor compound (e.g., an ITK or BTK inhibitor, such as, for example, ibrutinib) is administered prior to or concurrently with the allogeneic hematopoietic stem cells and/or allogeneic T-cells.

In the following description of irreversible BTK compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. In addition, nucleic acid and amino acid sequences for BTK (e.g., human BTK) are known in the art as disclosed in, e.g., U.S. Pat. No. 6,326,469. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

The BTK inhibitor compounds described herein are selective for BTK and kinases having a cysteine residue in an amino acid sequence position of the tyrosine kinase that is homologous to the amino acid sequence position of cysteine 481 in BTK. Generally, an irreversible inhibitor compound of BTK used in the methods described herein is identified or characterized in an in vitro assay, e.g., an acellular biochemical assay or a cellular functional assay. Such assays are useful to determine an in vitro IC₅₀ for an irreversible BTK inhibitor compound.

For example, an acellular kinase assay can be used to determine BTK activity after incubation of the kinase in the absence or presence of a range of concentrations of a candidate irreversible BTK inhibitor compound. If the candidate compound is in fact an irreversible BTK inhibitor, BTK kinase activity will not be recovered by repeat washing with inhibitor-free medium. See, e.g., J. B. Smaill, et al. (1999), J. Med. Chem. 42(10):1803-1815. Further, covalent complex formation between BTK and a candidate irreversible BTK inhibitor is a useful indicator of irreversible inhibition of BTK that can be readily determined by a number of methods known in the art (e.g., mass spectrometry). For example, some irreversible BTK-inhibitor compounds can form a covalent bond with Cys 481 of BTK (e.g., via a Michael reaction).

Cellular functional assays for BTK inhibition include measuring one or more cellular endpoints in response to stimulating a BTK-mediated pathway in a cell line (e.g., BCR activation in Ramos cells) in the absence or presence of a range of concentrations of a candidate irreversible BTK inhibitor compound. Useful endpoints for determining a response to BCR activation include, e.g., autophosphorylation of BTK, phosphorylation of a BTK target protein (e.g., PLC-γ), and cytoplasmic calcium flux.

High-throughput assays for many acellular biochemical assays (e.g., kinase assays) and cellular functional assays (e.g., calcium flux) are well known to those of ordinary skill in the art. In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. Automated systems thereby allow the identification and characterization of a large number of irreversible BTK compounds without undue effort.

In some embodiments, the BTK inhibitor is selected from the group consisting of a small organic molecule, a macromolecule, a peptide or a non-peptide.

In some embodiments, the BTK inhibitor provided herein is a reversible or irreversible inhibitor. In certain embodiments, the BTK inhibitor is an irreversible inhibitor.

In some embodiments, the irreversible BTK inhibitor forms a covalent bond with a cysteine sidechain of a Bruton's tyrosine kinase, a Bruton's tyrosine kinase homolog, or a BTK tyrosine kinase cysteine homolog.

Irreversible BTK inhibitor compounds can be used for the manufacture of a medicament for treating any of the foregoing conditions (e.g., autoimmune diseases, inflammatory diseases, allergy disorders, B-cell proliferative disorders, or thromboembolic disorders).

In some embodiments, the irreversible BTK inhibitor compound used for the methods described herein inhibits BTK or a BTK homolog kinase activity with an in vitro IC₅₀ of less than 10 μM (e.g., less than 1 μM, less than 0.5 μM, less than 0.4 μM, less than 0.3 μM, less than 0.1, less than 0.08 μM, less than 0.06 μM, less than 0.05 μM, less than 0.04 μM, less than 0.03 μM, less than less than 0.02 μM, less than 0.01, less than 0.008 μM, less than 0.006 μM, less than 0.005 μM, less than 0.004 μM, less than 0.003 μM, less than less than 0.002 μM, less than 0.001, less than 0.00099 M, less than 0.00098 μM, less than 0.00097 μM, less than 0.00096 μM, less than 0.00095 μM, less than 0.00094 μM, less than 0.00093 μM, less than 0.00092, or less than 0.00090 μM).

In some embodiments, the irreversible BTK inhibitor compound is selected from among ibrutinib (PCI-32765), PCI-45292, PCI-45466, AVL-101, AVL-291, AVL-292, or ONO-WG-37. In some embodiments, the irreversible BTK inhibitor compound is ibrutinib.

In one embodiment, the irreversible BTK inhibitor compound selectively and irreversibly inhibits an activated form of its target tyrosine kinase (e.g., a phosphorylated form of the tyrosine kinase). For example, activated BTK is transphosphorylated at tyrosine 551. Thus, in these embodiments the irreversible BTK inhibitor inhibits the target kinase in cells only once the target kinase is activated by the signaling events.

In other embodiments, the BTK inhibitor used in the methods describe herein has the structure of any of Formula (A). Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided.

Definition of standard chemistry terms are found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques are optionally used for chemical syntheses, chemical analyses, pharmaceutical preparation, formulation, and delivery, and treatment of patients. Standard techniques are optionally used for recombinant DNA, oligonucleotide synthesis, and tissue culture and transformation (e.g., electroporation, lipofection). Reactions and purification techniques are performed using documented methodologies or as described herein.

It is to be understood that the methods and compositions described herein are not limited to the particular methodology, protocols, cell lines, constructs, and reagents described herein and as such optionally vary. It is also to be understood that the terminology used herein is for the purpose of describing particular embodiments only, and is not intended to limit the scope of the methods and compositions described herein, which will be limited only by the appended claims.

Unless stated otherwise, the terms used for complex moieties (i.e., multiple chains of moieties) are to be read equivalently either from left to right or right to left. For example, the group alkylenecycloalkylene refers both to an alkylene group followed by a cycloalkylene group or as a cycloalkylene group followed by an alkylene group.

The suffix “ene” appended to a group indicates that such a group is a diradical. By way of example only, a methylene is a diradical of a methyl group, that is, it is a —CH₂— group; and an ethylene is a diradical of an ethyl group, i.e., —CH₂CH₂—.

An “alkyl” group refers to an aliphatic hydrocarbon group. The alkyl moiety includes a “saturated alkyl” group, which means that it does not contain any alkene or alkyne moieties. The alkyl moiety also includes an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, includes branched, straight chain, or cyclic moieties. Depending on the structure, an alkyl group includes a monoradical or a diradical (i.e., an alkylene group), and if a “lower alkyl” having 1 to 6 carbon atoms.

As used herein, C₁-Cₓ includes C₁-C₂, C₁-C₃ . . . C₁-Cₓ.

The “alkyl” moiety optionally has 1 to 10 carbon atoms (whenever it appears herein, a numerical range such as “1 to 10” refers to each integer in the given range; e.g., “1 to 10 carbon atoms” means that the alkyl group is selected from a moiety having 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, isobutyl, sec-butyl, and t-butyl. Thus C₁-C₄ alkyl includes C₁-C₂ alkyl and C₁-C₃ alkyl. Alkyl groups are optionally substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which are either the same or different. The alkenyl moiety is optionally branched, straight chain, or cyclic (in which case, it is also known as a “cycloalkenyl” group). Depending on the structure, an alkenyl group includes a monoradical or a diradical (i.e., an alkenylene group). Alkenyl groups are optionally substituted. Non-limiting examples of an alkenyl group include —CH═CH₂, —C(CH₃)═CH₂, —CH═CHCH₃, —C(CH₃)═CHCH₃. Alkenylene groups include, but are not limited to, —CH═CH—, —C(CH₃)═CH—, —CH═CHCH₂—, —CH═CHCH₂CH₂— and —C(CH₃)═CHCH₂—. Alkenyl groups optionally have 2 to 10 carbons, and if a “lower alkenyl” having 2 to 6 carbon atoms.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which is either the same or different. The “R” portion of the alkynyl moiety may be branched, straight chain, or cyclic. Depending on the structure, an alkynyl group includes a monoradical or a diradical (i.e., an alkynylene group). Alkynyl groups are optionally substituted. Non-limiting examples of an alkynyl group include, but are not limited to, —C≡CH, —C≡CCH₃, —C≡CCH₂CH₃, —C≡C—, and —C≡CCH₂—. Alkynyl groups optionally have 2 to 10 carbons, and if a “lower alkynyl” having 2 to 6 carbon atoms.

An “alkoxy” group refers to a (alkyl)O— group, where alkyl is as defined herein.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, optionally form a cyclic ring system.

“Alkylaminoalkyl” refers to an alkyl radical, as defined herein, substituted with an alkylamine, as defined herein.

“Hydroxyalkylaminoalkyl” refers to an alkyl radical, as defined herein, substituted with an alkylamine, and alkylhydroxy, as defined herein.

“Alkoxyalkylaminoalkyl” refers to an alkyl radical, as defined herein, substituted with an alkylamine and substituted with an alkylalkoxy, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). In some embodiments, an amide moiety forms a linkage between an amino acid or a peptide molecule and a compound described herein, thereby forming a prodrug. Any amine, or carboxyl side chain on the compounds described herein can be amidified. The procedures and specific groups to make such amides are found in sources such as Greene and Wuts, Protective Groups in Organic Synthesis, 3^{rd} Ed., John Wiley & Sons, New York, N.Y., 1999, which is incorporated herein by reference for this disclosure.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are found in sources such as Greene and Wuts, Protective Groups in Organic Synthesis, 3^{rd} Ed., John Wiley & Sons, New York, N.Y., 1999, which is incorporated herein by reference for this disclosure.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e. a heteroatom). Heterocycle includes heteroaryl and heterocycloalkyl. Carbocycles and heterocycles can be optionally substituted.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

The term “carbonyl” as used herein refers to a group containing a moiety selected from the group consisting of —C(O)—, —S(O)—, —S(O)2-, and —C(S)—, including, but not limited to, groups containing a least one ketone group, and/or at least one aldehyde group, and/or at least one ester group, and/or at least one carboxylic acid group, and/or at least one thioester group. Such carbonyl groups include ketones, aldehydes, carboxylic acids, esters, and thioesters. In some embodiments, such groups are a part of linear, branched, or cyclic molecules.

The term “cycloalkyl” refers to a monocyclic or polycyclic radical that contains only carbon and hydrogen, and is optionally saturated, partially unsaturated, or fully unsaturated. Cycloalkyl groups include groups having from 3 to 10 ring atoms. Illustrative examples of cycloalkyl groups include the following moieties:

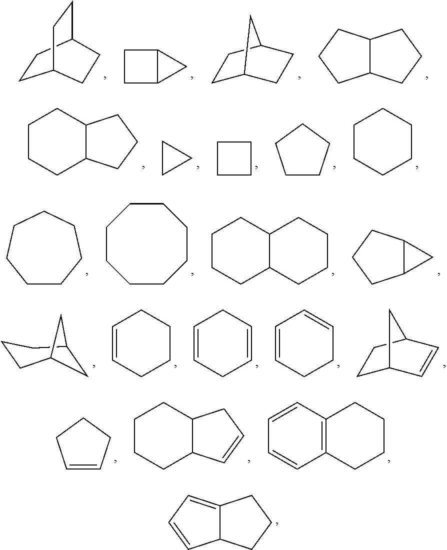

and the like. Depending on the structure, a cycloalkyl group is either a monoradical or a diradical (e.g., an cycloalkylene group), and if a “lower cycloalkyl” having 3 to 8 carbon atoms.

“Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, are optionally C-attached or N-attached where such is possible. For instance, a group derived from pyrrole includes pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The terms “heteroaryl” or, alternatively, “heteroaromatic” refers to an aromatic group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Illustrative examples of heteroaryl groups include the following moieties:

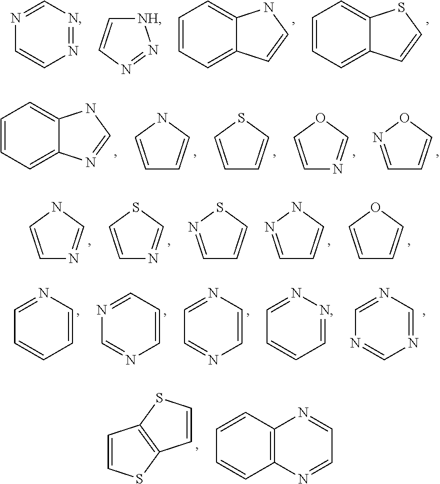

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. In some embodiments, the radicals are fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

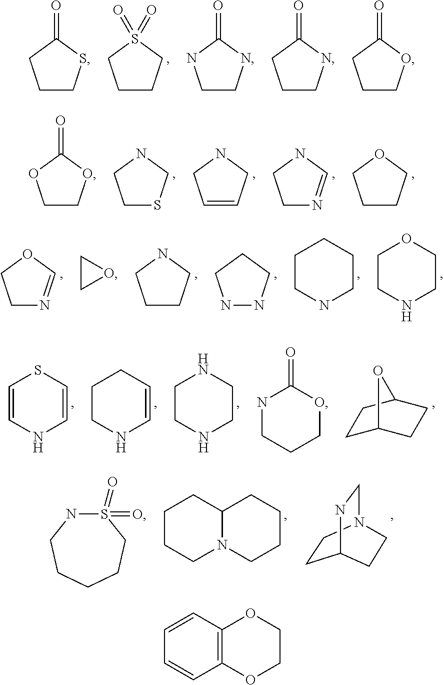

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

The term “halo” or, alternatively, “halogen” or “halide” means fluoro, chloro, bromo, and iodo.

The term “haloalkyl,” refers to alkyl structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

The term “fluoroalkyl,” as used herein, refers to alkyl group in which at least one hydrogen is replaced with a fluorine atom. Examples of fluoroalkyl groups include, but are not limited to, —CF₃, —CH₂CF₃, —CF₂CF₃, —CH₂CH₂CF₃ and the like.

As used herein, the term “heteroalkyl” refers to optionally substituted alkyl radicals in which one or more skeletal chain atoms is a heteroatom, e.g., oxygen, nitrogen, sulfur, silicon, phosphorus or combinations thereof. The heteroatom(s) are placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, in some embodiments, up to two heteroatoms are consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “SH” group is also referred to either as a thiol group or a sulfhydryl group.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted C₁-C₄alkyl), (substituted or unsubstituted C₃-C₆cycloalkyl), heteroaryl, or heteroalkyl. The protecting groups that form the protective derivatives of the above substituents include those found in sources such as Greene and Wuts, above.

### ACK Inhibitor Compounds

Described herein, in some embodiments, are method of treating alloantibody driven chronic graft versus host disease (cGVHD) in a patient in need thereof, comprising administering a therapeutically effective amount of an ACK inhibitor (e.g., an ITK or BTK inhibitor).

Further described herein are methods of preventing the occurrence of graft versus host disease (cGVHD) or reducing the severity of cGVHD occurrence in a patient requiring cell transplantation comprising administering to the patient a composition comprising a therapeutically-effective amount of an ACK inhibitor compound (e.g., an ITK or BTK inhibitor, such as, for example, ibrutinib).

Further described herein are methods of treating a patient for alleviation of a bone marrow mediated disease, with alleviation of consequently developed graft versus host disease (cGVHD), comprising administering to the patient allogeneic hematopoietic stem cells and/or allogeneic T-cells, wherein a therapeutically effective amount of an ACK inhibitor compound (e.g., an ITK or BTK inhibitor, such as, for example, ibrutinib) is administered prior to or concurrently with the allogeneic hematopoietic stem cells and/or allogeneic T-cells.

The ACK inhibitor compounds described herein are selective for kinases having an accessible cysteine that is able to form a covalent bond with a Michael acceptor moiety on the inhibitor compound. In some embodiments, the cysteine residue is accessible or becomes accessible when the binding site moiety of the irreversible inhibitor binds to the kinase. That is, the binding site moiety of the irreversible inhibitor binds to an active site of the ACK and the Michael acceptor moiety of irreversible inhibitor gains access (in one embodiment the step of binding leads to a conformational change in the ACK, thus exposing the cysteine) or is otherwise exposed to the cysteine residue of the ACK; as a result a covalent bond is formed between the “S” of the cysteine residue and the Michael acceptor of the irreversible inhibitor. Consequently, the binding site moiety of the irreversible inhibitor remains bound or otherwise blocks the active site of the ACK.

In some embodiments, the ACK is BTK, a homolog of BTK or a tyrosine kinase having a cysteine residue in an amino acid sequence position that is homologous to the amino acid sequence position of cysteine 481 in BTK. In some embodiments, the ACK is ITK. In some embodiments, the ACK is HER4. Inhibitor compounds described herein include a Michael acceptor moiety, a binding site moiety and a linker that links the binding site moiety and the Michael acceptor moiety (and in some embodiments, the structure of the linker provides a conformation, or otherwise directs the Michael acceptor moiety, so as to improve the selectivity of the irreversible inhibitor for a particular ACK). In some embodiments, the ACK inhibitor inhibits ITK and BTK.

In some embodiments, the ACK inhibitor is a compound of Formula (A):

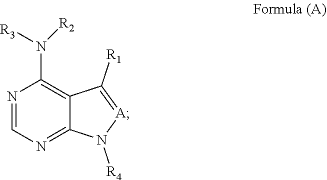

wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
R₂ and R₃ are independently selected from H, lower alkyl and substituted lower alkyl;
R₄ is L₃-X-L₄-G, wherein,
   L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L₃, X and L₄ taken together form a nitrogen containing heterocyclic ring;
   G is

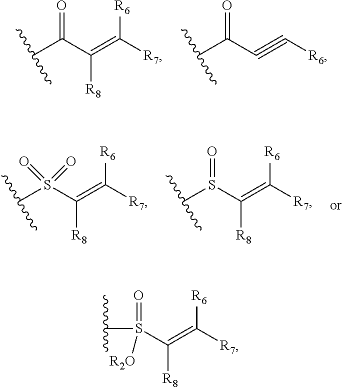

wherein,
   R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and pharmaceutically active metabolites, pharmaceutically acceptable solvates, pharmaceutically acceptable salts, or pharmaceutically acceptable prodrugs thereof.

In some embodiments, the compound of Formula (A) is a BTK inhibitor. In some embodiments, the compound of Formula (A) is an ITK inhibitor. In some embodiments, the compound of Formula (A) inhibits ITK and BTK. In some embodiments, the compound of Formula (A) has the structure:

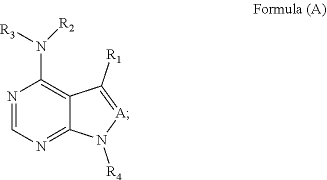

wherein:
A is N;
R₂ and R₃ are each H;
R₁ is phenyl-O-phenyl or phenyl-S-phenyl; and
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;
G is

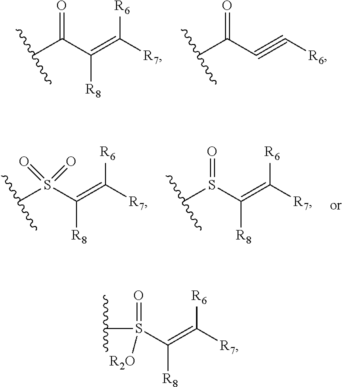

wherein
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl.

In some embodiments, the ACK inhibitor is (R)-1-(3-(4-amino-3-(4-phenoxyphenyl)-1H-pyrazolo[3,4-d]pyrimidin-1-yl)piperidin-1-yl)prop-2-en-1-one (i.e. PCI-32765/ibrutinib)

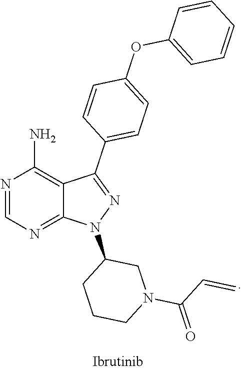 [SMILES: C=CC(=O)N1CCC[C@@H](n2nc(-c3ccc(Oc4ccccc4)cc3)c3c(N)ncnc32)C1]

In some embodiments, the ACK inhibitor is ibrutinib, PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) or JTE-051 (Japan Tobacco Inc).

In some embodiments, the ACK inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066).

In some embodiments, the ACK inhibitor is:

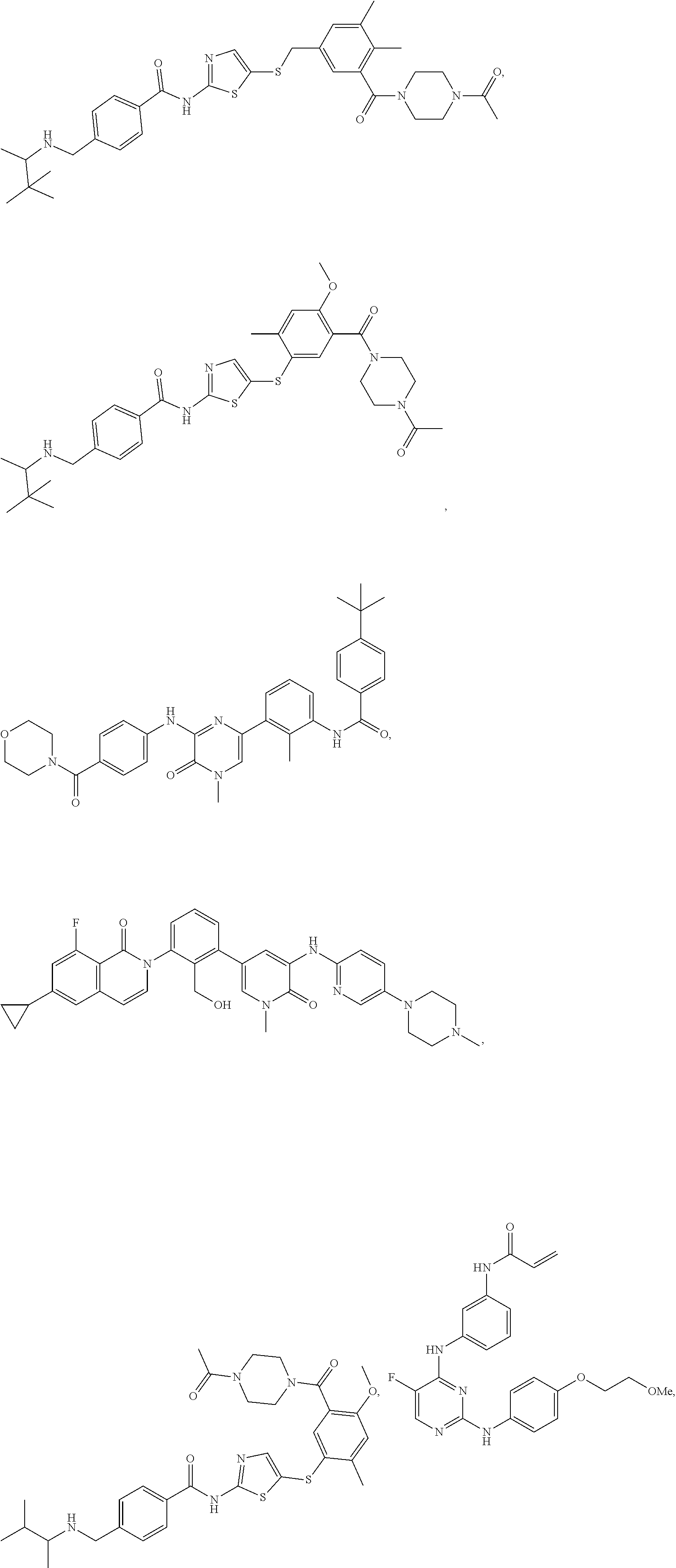

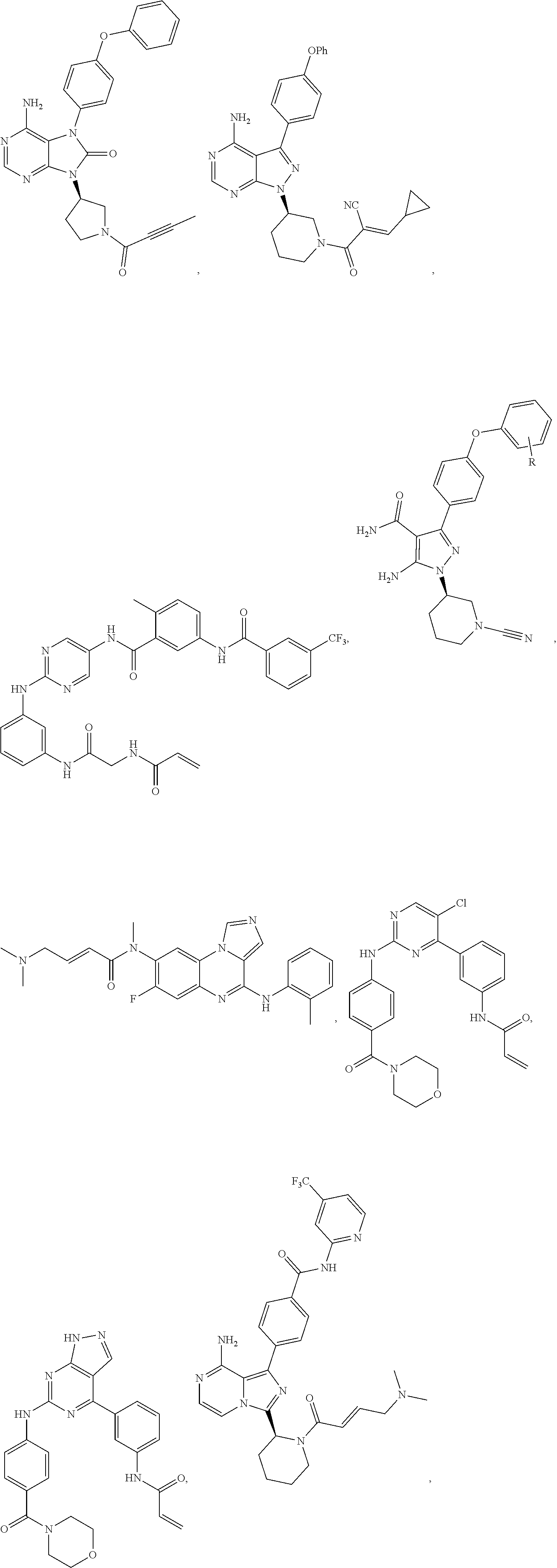

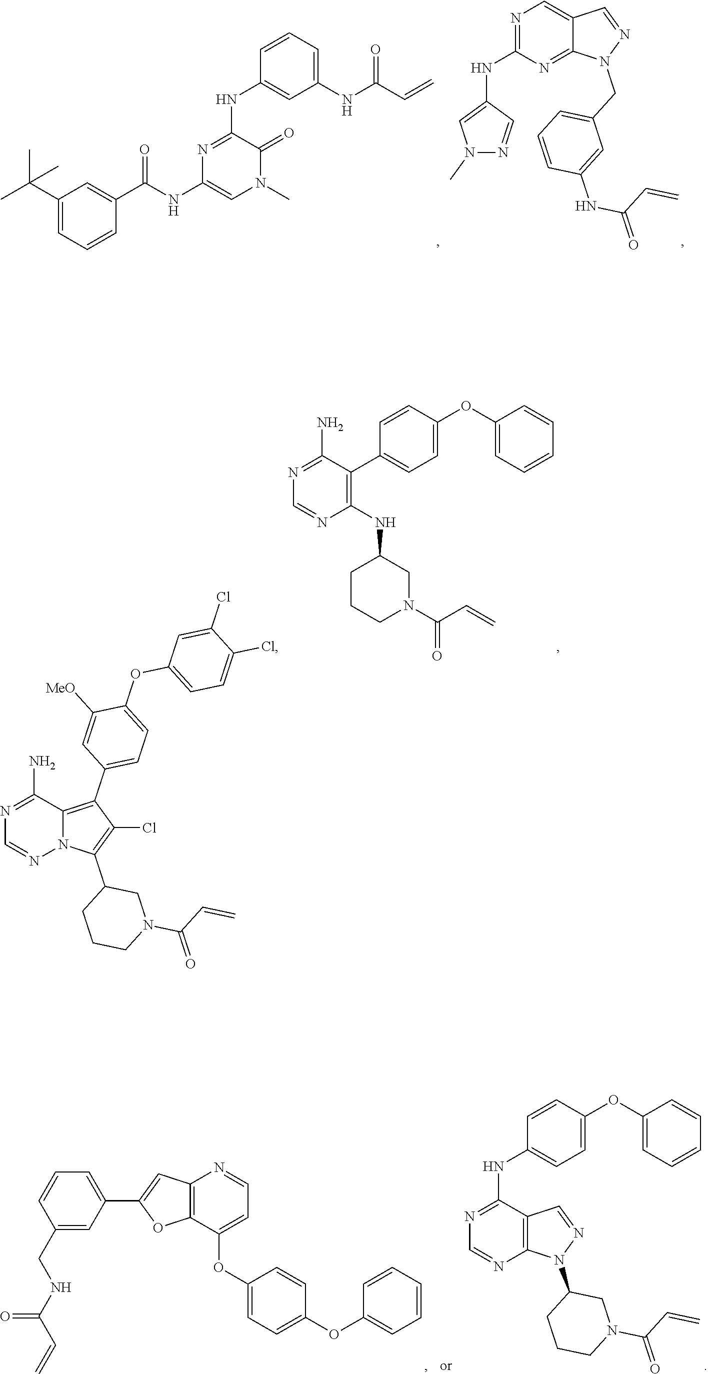

### BTK Inhibitors

In some embodiments, the ACK inhibitor is a BTK inhibitor. The BTK inhibitor compounds described herein are selective for BTK and kinases having a cysteine residue in an amino acid sequence position of the tyrosine kinase that is homologous to the amino acid sequence position of cysteine 481 in BTK. The BTK inhibitor compound can form a covalent bond with Cys 481 of BTK (e.g., via a Michael reaction).

In some embodiments, the BTK inhibitor is a compound of Formula (A) having the structure:

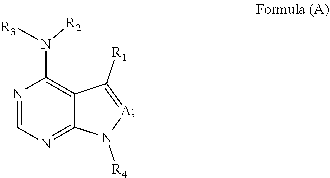

wherein:

### A is N;

R₁ is phenyl-O-phenyl or phenyl-S-phenyl;
R₂ and R₃ are independently H;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;

### G is

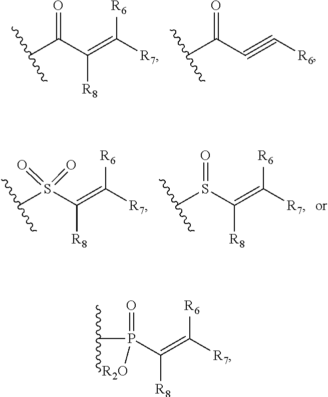

wherein,
R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

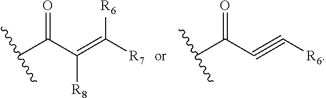

In some embodiments, the compound of Formula (A) is 1-[(3R)-3-[4-amino-3-(4-phenoxyphenyl)pyrazolo[3,4-d]pyrimidin-1-yl]piperidin-1-yl]prop-2-en-1-one.

In some embodiments, the BTK inhibitor compound of Formula (A) has the following structure of Formula (B):

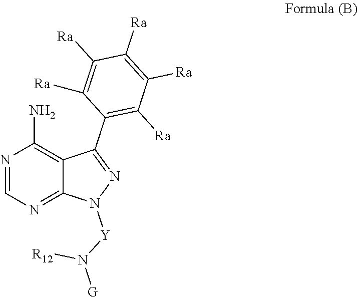

wherein:
Y is alkyl or substituted alkyl, or a 4-, 5-, or 6-membered cycloalkyl ring;
each Rₐ is independently H, halogen, —CF₃, —CN, —NO₂, OH, NH₂, -Lₐ-(substituted or unsubstituted alkyl), -Lₐ-(substituted or unsubstituted alkenyl), -Lₐ-(substituted or unsubstituted heteroaryl), or -Lₐ-(substituted or unsubstituted aryl), wherein Lₐ is a bond, O, S, —S(═O), —S(═O)₂, NH, C(O), CH₂, —NHC(O)O, —NHC(O), or —C(O)NH;

### G is

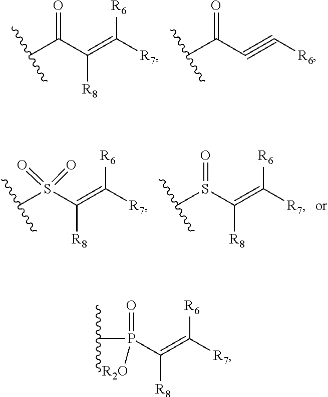

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₁₂ is H or lower alkyl; or
Y and R₁₂ taken together form a 4-, 5-, or 6-membered heterocyclic ring; and
pharmaceutically acceptable active metabolites, pharmaceutically acceptable solvates, pharmaceutically acceptable salts, or pharmaceutically acceptable prodrugs thereof.

In some embodiments, G is selected from among

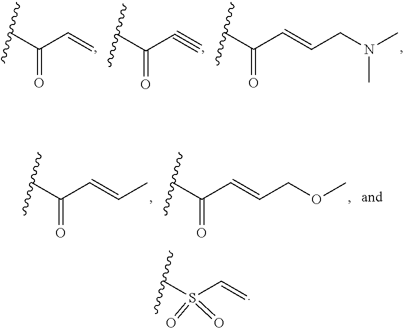

In some embodiments,

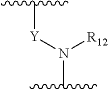

is selected from among

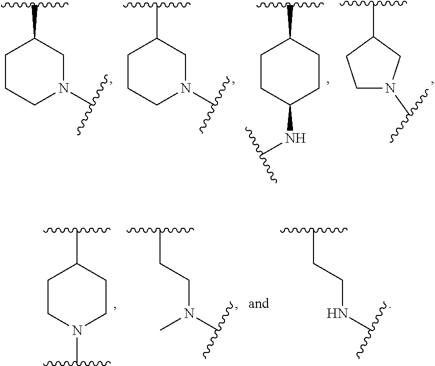

In some embodiments, the BTK inhibitor compound of Formula (B) has the following structure of Formula (C):

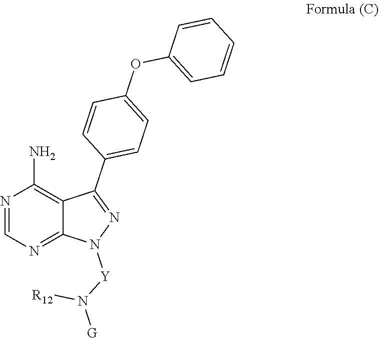

Y is alkyl or substituted alkyl, or a 4-, 5-, or 6-membered cycloalkyl ring;
R₁₂ is H or lower alkyl; or
Y and R₁₂ taken together form a 4-, 5-, or 6-membered heterocyclic ring;

### G is

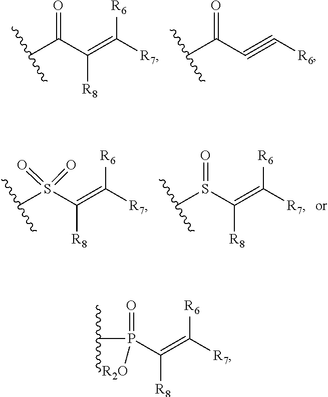

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl; and
pharmaceutically acceptable active metabolites, pharmaceutically acceptable solvates, pharmaceutically acceptable salts, or pharmaceutically acceptable prodrugs thereof.

In some embodiments, the “G” group of any of Formula (A), Formula (B), or Formula (C) is any group that is used to tailor the physical and biological properties of the molecule. Such tailoring/modifications are achieved using groups which modulate Michael acceptor chemical reactivity, acidity, basicity, lipophilicity, solubility and other physical properties of the molecule. The physical and biological properties modulated by such modifications to G include, by way of example only, enhancing chemical reactivity of Michael acceptor group, solubility, in vivo absorption, and in vivo metabolism. In addition, in vivo metabolism may include, by way of example only, controlling in vivo PK properties, off-target activities, potential toxicities associated with cypP450 interactions, drug-drug interactions, and the like. Further, modifications to G allow for the tailoring of the in vivo efficacy of the compound through the modulation of, by way of example, specific and non-specific protein binding to plasma proteins and lipids and tissue distribution in vivo.

In some embodiments, the BTK inhibitor has the structure of Formula (D):

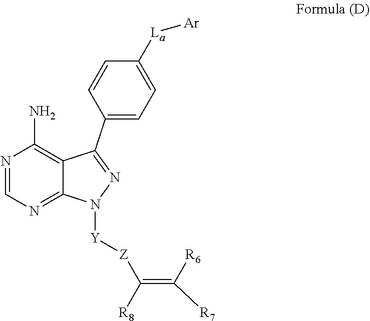

wherein
Lₐ is CH₂, O, NH or S;
Ar is an optionally substituted aromatic carbocycle or an aromatic heterocycle;
Y is an optionally substituted alkyl, heteroalkyl, carbocycle, heterocycle, or combination thereof;
Z is C(O), OC(O), NHC(O), C(S), S(O)ₓ, OS(O)ₓ, NHS(O)ₓ, where x is 1 or 2; and
R₆, R₇, and R₈ are independently selected from H, alkyl, heteroalkyl, carbocycle, heterocycle, or combinations thereof.

In some embodiments, Lₐ is O.

In some embodiments, Ar is phenyl.

In some embodiments, Z is C(O).

In some embodiments, each of R₁, R₂, and R₃ is H.

In some embodiments, provided herein is a compound of Formula (D). Formula (D) is as follows:

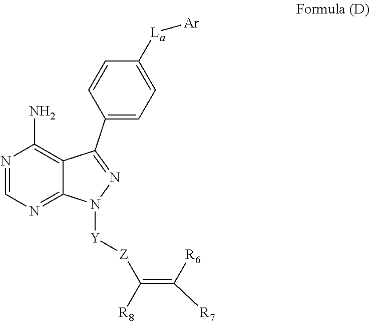

wherein:
Lₐ is CH₂, O, NH or S;
Ar is a substituted or unsubstituted aryl, or a substituted or unsubstituted heteroaryl;
Y is an optionally substituted group selected from among alkyl, heteroalkyl, cycloalkyl, heterocycloalkyl, aryl, and heteroaryl;
Z is C(═O), OC(═O), NHC(═O), C(═S), S(═O)ₓ, OS(═O)ₓ, NHS(═O)ₓ, where x is 1 or 2;
R₇ and R₈ are independently selected from among H, unsubstituted C₁-C₄alkyl, substituted C₁-C₄alkyl, unsubstituted C₁-C₄heteroalkyl, substituted C₁-C₄heteroalkyl, unsubstituted C₃-C₆cycloalkyl, substituted C₃-C₆cycloalkyl, unsubstituted C₂-C₆heterocycloalkyl, and substituted C₂-C₆heterocycloalkyl; or
R₇ and R₈ taken together form a bond;
R₆ is H, substituted or unsubstituted C₁-C₄alkyl, substituted or unsubstituted C₁-C₄heteroalkyl, C₁-C₆alkoxyalkyl, C₁-C₈alkylaminoalkyl, substituted or unsubstituted C₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl); and
pharmaceutically active metabolites, or pharmaceutically acceptable solvates, pharmaceutically acceptable salts, or pharmaceutically acceptable prodrugs thereof.

For any and all of the embodiments, substituents can be selected from among from a subset of the listed alternatives. For example, in some embodiments, Lₐ is CH₂, O, or NH. In other embodiments, Lₐ is O or NH. In yet other embodiments, Lₐ is O.

In some embodiments, Ar is a substituted or unsubstituted aryl. In yet other embodiments, Ar is a 6-membered aryl. In some other embodiments, Ar is phenyl.

In some embodiments, x is 2. In yet other embodiments, Z is C(═O), OC(═O), NHC(═O), S(═O)ₓ, OS(═O)ₓ, or NHS(═O)ₓ. In some other embodiments, Z is C(═O), NHC(═O), or S(═O)₂.

In some embodiments, R₇ and R₈ are independently selected from among H, unsubstituted C₁-C₄ alkyl, substituted C₁-C₄alkyl, unsubstituted C₁-C₄heteroalkyl, and substituted C₁-C₄heteroalkyl; or R₇ and R₈ taken together form a bond. In yet other embodiments, each of R₇ and R₈ is H; or R₇ and R₈ taken together form a bond.

In some embodiments, R₆ is H, substituted or unsubstituted C₁-C₄alkyl, substituted or unsubstituted C₁-C₄heteroalkyl, C₁-C₆alkoxyalkyl, C₁-C₂alkyl-N(C₁-C₃alkyl)₂, substituted or unsubstituted aryl, substituted or unsubstituted heteroaryl, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl). In some other embodiments, R₆ is H, substituted or unsubstituted C₁-C₄alkyl, substituted or unsubstituted C₁-C₄heteroalkyl, C₁-C₆alkoxyalkyl, C₁-C₂alkyl-N(C₁-C₃alkyl)₂, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl). In yet other embodiments, R₆ is H, substituted or unsubstituted C₁-C₄alkyl, —CH₂—O—(C₁-C₃alkyl), —CH₂—N(C₁-C₃alkyl)₂, C₁-C₄alkyl(phenyl), or C₁-C₄alkyl(5- or 6-membered heteroaryl). In some embodiments, R₆ is H, substituted or unsubstituted C₁-C₄alkyl, —CH₂—O—(C₁-C₃alkyl), —CH₂—N(C₁-C₃alkyl)₂, C₁-C₄alkyl(phenyl), or C₁-C₄alkyl(5- or 6-membered heteroaryl containing 1 or 2 N atoms), or C₁-C₄alkyl(5- or 6-membered heterocycloalkyl containing 1 or 2 N atoms).

In some embodiments, Y is an optionally substituted group selected from among alkyl, heteroalkyl, cycloalkyl, and heterocycloalkyl. In other embodiments, Y is an optionally substituted group selected from among C₁-C₆alkyl, C₁-C₆heteroalkyl, 4-, 5-, 6- or 7-membered cycloalkyl, and 4-, 5-, 6- or 7-membered heterocycloalkyl. In yet other embodiments, Y is an optionally substituted group selected from among C₁-C₆alkyl, C₁-C₆heteroalkyl, 5-, or 6-membered cycloalkyl, and 5-, or 6-membered heterocycloalkyl containing 1 or 2 N atoms. In some other embodiments, Y is a 5-, or 6-membered cycloalkyl, or a 5-, or 6-membered heterocycloalkyl containing 1 or 2 N atoms.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments the BTK inhibitor compounds of Formula (A), Formula (B), Formula (C), Formula (D), include, but are not limited to, compounds selected from the group consisting of:

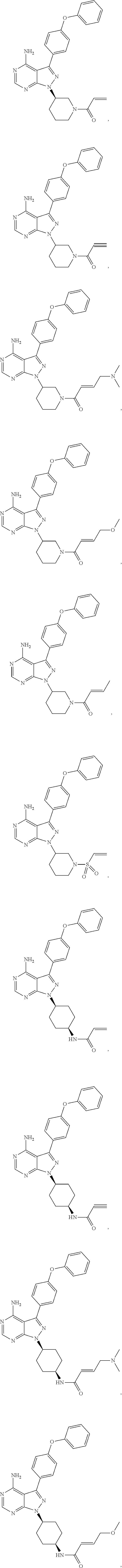

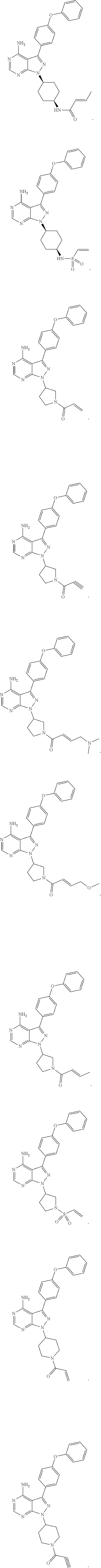

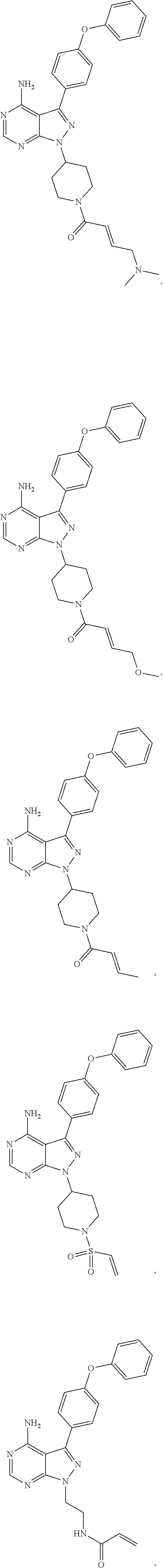 [SMILES: CN(C)C/C=C/C(=O)N1CCC(n2nc(-c3ccc(Oc4ccccc4)cc3)c3c(N)ncnc32)CC1 ; COC/C=C/C(=O)N1CCC(n2nc(-c3ccc(Oc4ccccc4)cc3)c3c(N)ncnc32)CC1 ; C/C=C/C(=O)N1CCC(n2nc(-c3ccc(Oc4ccccc4)cc3)c3c(N)ncnc32)CC1 ; C=CS(=O)(=O)N1CCC(n2nc(-c3ccc(Oc4ccccc4)cc3)c3c(N)ncnc32)CC1 ; C=CC(=O)NCCn1nc(-c2ccc(Oc3ccccc3)cc2)c2c(N)ncnc21]

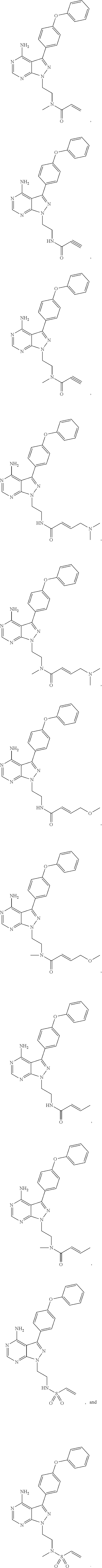

In some embodiments, the BTK inhibitor compounds are selected from among:

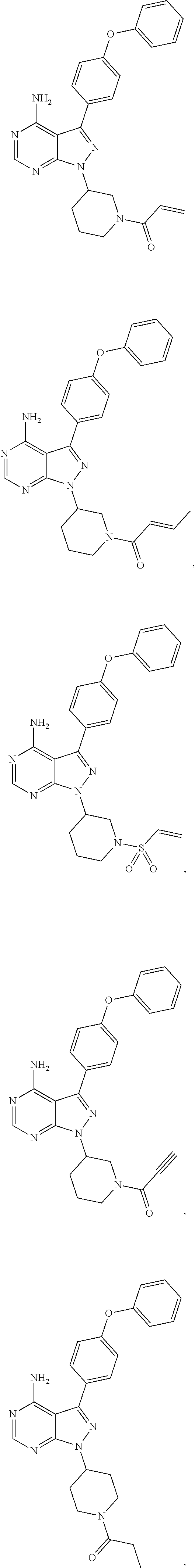 [SMILES: C=CC(=O)N1CCCC(n2nc(-c3ccc(Oc4ccccc4)cc3)c3c(N)ncnc32)C1 ; C/C=C/C(=O)N1CCCC(n2nc(-c3ccc(Oc4ccccc4)cc3)c3c(N)ncnc32)C1 ; C=CS(=O)(=O)N1CCCC(n2nc(-c3ccc(Oc4ccccc4)cc3)c3c(N)ncnc32)C1 ; C#CC(=O)N1CCCC(n2nc(-c3ccc(Oc4ccccc4)cc3)c3c(N)ncnc32)C1 ; CCC(=O)N1CCC(n2nc(-c3ccc(Oc4ccccc4)cc3)c3c(N)ncnc32)CC1]

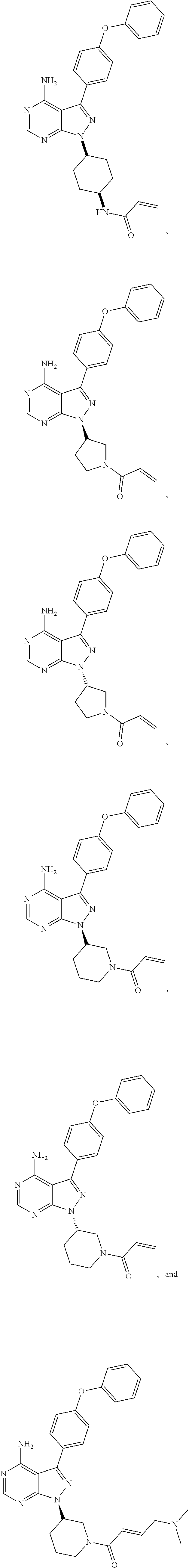

In some embodiments, the BTK inhibitor compounds are selected from among: 1-(3-(4-amino-3-(4-phenoxyphenyl)-1H-pyrazolo[3,4-d]pyrimidin-1-yl)piperidin-1-yl)prop-2-en-1-one (Compound 4); (E)-1-(3-(4-amino-3-(4-phenoxyphenyl)-1H-pyrazolo[3,4-d]pyrimidin-1-yl)piperidin-1-yl)but-2-en-1-one (Compound 5); 1-(3-(4-amino-3-(4-phenoxyphenyl)-1H-pyrazolo[3,4-d]pyrimidin-1-yl)piperidin-1-yl)sulfonylethene (Compound 6); 1-(3-(4-amino-3-(4-phenoxyphenyl)-1H-pyrazolo[3,4-d]pyrimidin-1-yl)piperidin-1-yl)prop-2-yn-1-one (Compound 8); 1-(4-(4-amino-3-(4-phenoxyphenyl)-1H-pyrazolo[3,4-d]pyrimidin-1-yl)piperidin-1-yl)prop-2-en-1-one (Compound 9); N-((1s,4s)-4-(4-amino-3-(4-phenoxyphenyl)-1H-pyrazolo[3,4-d]pyrimidin-1-yl)cyclohexyl)acrylamide (Compound 10); 1-((R)-3-(4-amino-3-(4-phenoxyphenyl)-1H-pyrazolo[3,4-d]pyrimidin-1-yl)pyrrolidin-1-yl)prop-2-en-1-one (Compound 11); 1-((S)-3-(4-amino-3-(4-phenoxyphenyl)-1H-pyrazolo[3,4-d]pyrimidin-1-yl)pyrrolidin-1-yl)prop-2-en-1-one (Compound 12); 1-((R)-3-(4-amino-3-(4-phenoxyphenyl)-1H-pyrazolo[3,4-d]pyrimidin-1-yl)piperidin-1-yl)prop-2-en-1-one (Compound 13); 1-((S)-3-(4-amino-3-(4-phenoxyphenyl)-1H-pyrazolo[3,4-d]pyrimidin-1-yl)piperidin-1-yl)prop-2-en-1-one (Compound 14); and (E)-1-(3-(4-amino-3-(4-phenoxyphenyl)-1H-pyrazolo[3,4-d]pyrimidin-1-yl)piperidin-1-yl)-4-(dimethylamino)but-2-en-1-one (Compound 15).

Throughout the specification, groups and substituents thereof can be chosen by one skilled in the field to provide stable moieties and compounds.

The compounds of any of Formula (A), or Formula (B), or Formula (C), or Formula (D) can irreversibly inhibit Btk and may be used to treat patients suffering from Bruton's tyrosine kinase-dependent or Bruton's tyrosine kinase mediated conditions or diseases, including, but not limited to, cancer, autoimmune and other inflammatory diseases.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or Ibrutinib or any other suitable name refers to the compound with the following structure:

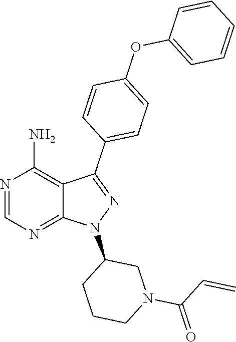 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from Ibrutinib and includes:

acid addition salts formed by reacting Ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;

acid addition salts formed by reacting Ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Ibrutinib refers to a salt of Ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Ibrutinib, or pharmaceutically acceptable salts thereof, are conveniently prepared or formed during the processes described herein. In some embodiments, solvates of Ibrutinib are anhydrous. In some embodiments, Ibrutinib, or pharmaceutically acceptable salts thereof, exist in unsolvated form. In some embodiments, Ibrutinib, or pharmaceutically acceptable salts thereof, exist in unsolvated form and are anhydrous.

In yet other embodiments, Ibrutinib, or a pharmaceutically acceptable salt thereof, is prepared in various forms, including but not limited to, amorphous phase, crystalline forms, milled forms and nano-particulate forms. In some embodiments, Ibrutinib, or a pharmaceutically acceptable salt thereof, is amorphous. In some embodiments, Ibrutinib, or a pharmaceutically acceptable salt thereof, is amorphous and anhydrous. In some embodiments, Ibrutinib, or a pharmaceutically acceptable salt thereof, is crystalline. In some embodiments, Ibrutinib, or a pharmaceutically acceptable salt thereof, is crystalline and anhydrous.

In some embodiments, Ibrutinib is prepared as outlined in U.S. Pat. No. 7,514,444.

In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) and JTE-051 (Japan Tobacco Inc).

In some embodiments, the BTK inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the BTK inhibitor is:

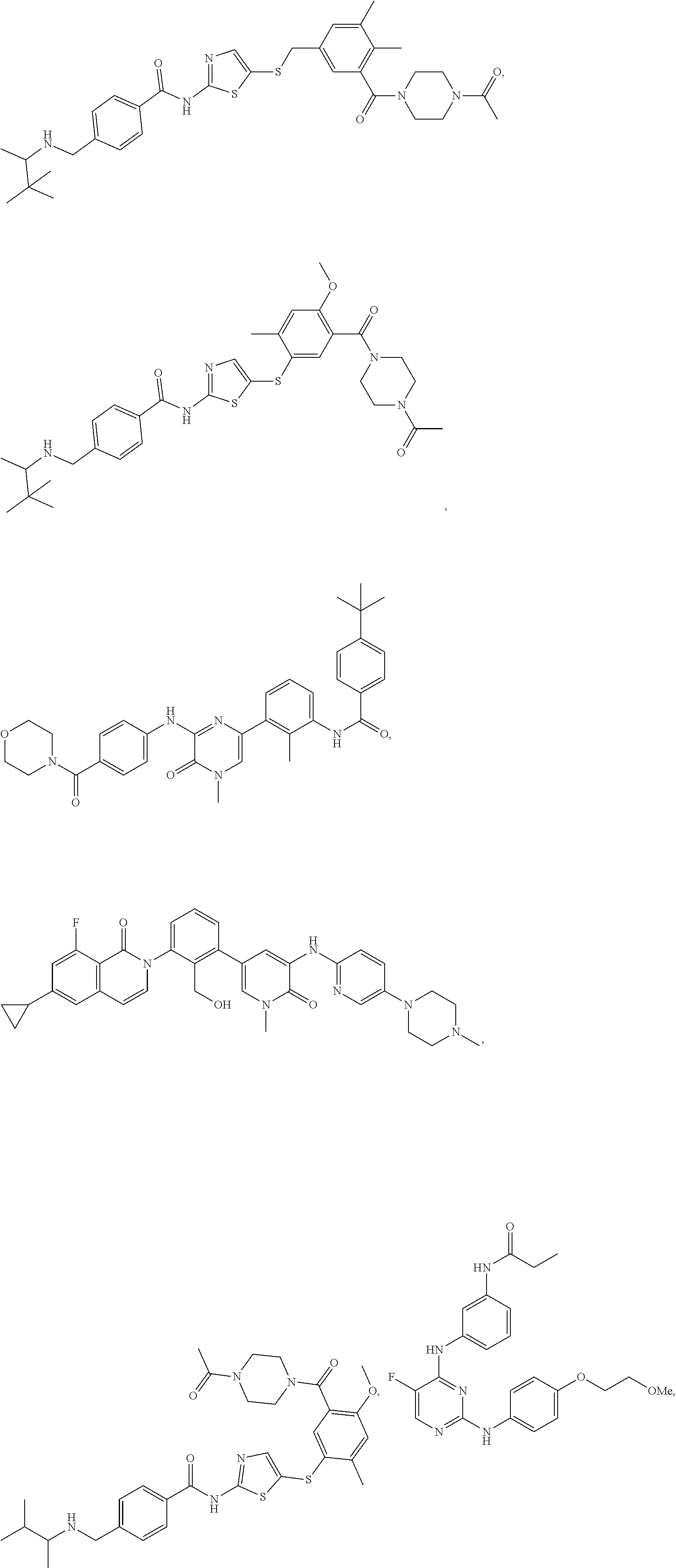

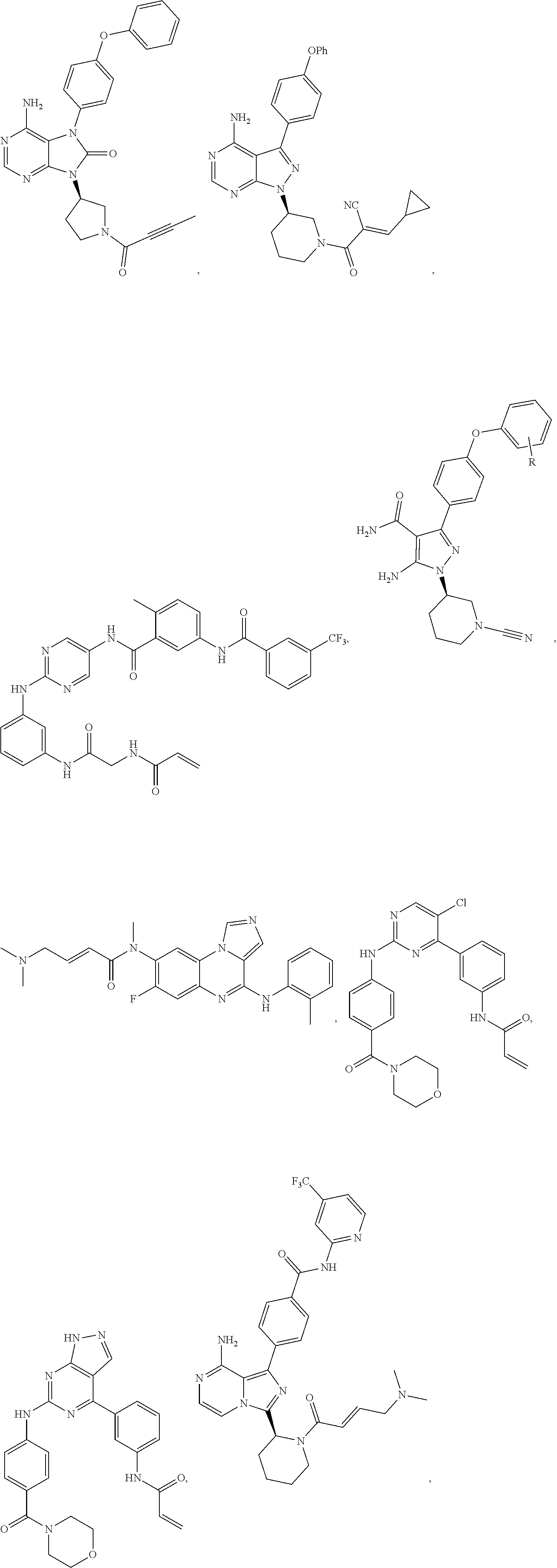

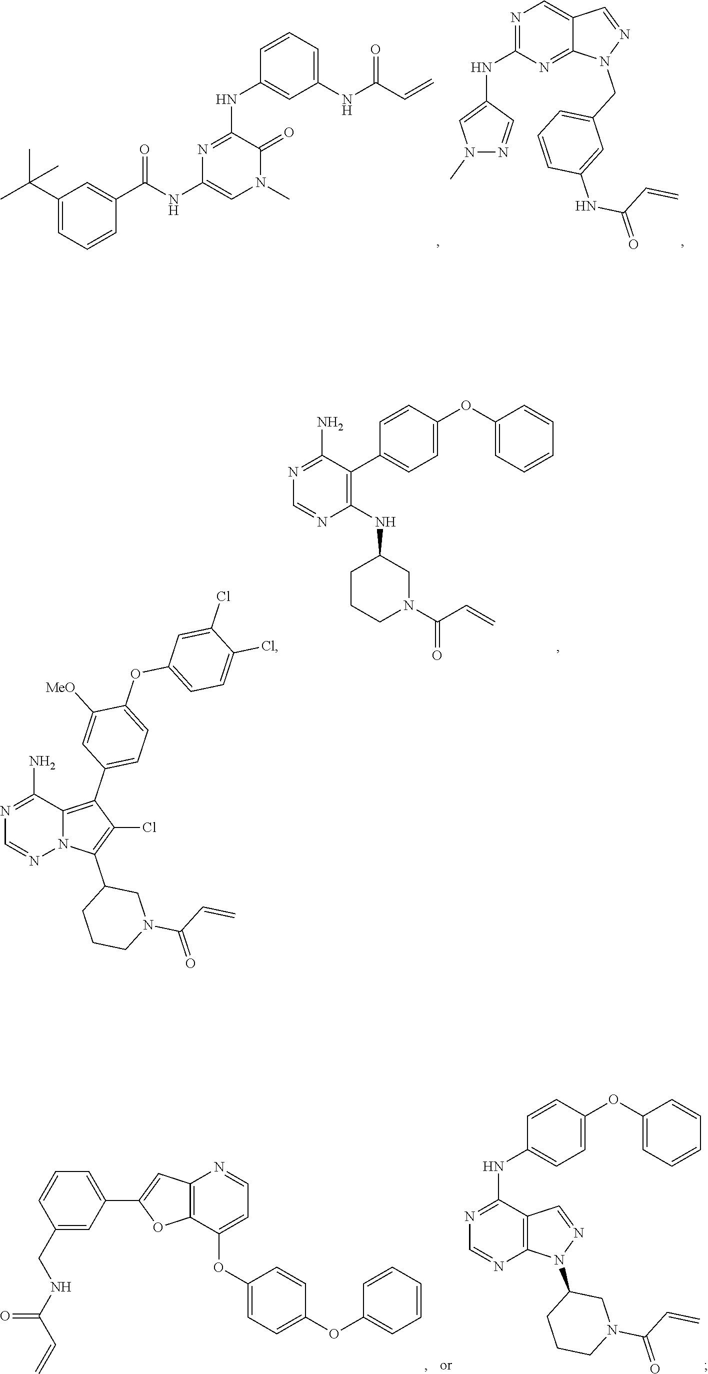

or a pharmaceutically acceptable salt thereof.

### ITK Inhibitors

In some embodiments, ACK inhibitor is an ITK inhibitor. In some embodiments, the ITK inhibitor covalently binds to Cysteine 442 of ITK. In some embodiments, the ITK inhibitor is an ITK inhibitor compound described in WO2002/0500071, which is incorporated by reference in its entirety. In some embodiments, the ITK inhibitor is an ITK inhibitor compound described in WO2005/070420, which is incorporated by reference in its entirety. In some embodiments, the ITK inhibitor is an ITK inhibitor compound described in WO2005/079791, which is incorporated by reference in its entirety. In some embodiments, the ITK inhibitor is an ITK inhibitor compound described in WO2007/076228, which is incorporated by reference in its entirety. In some embodiments, the ITK inhibitor is an ITK inhibitor compound described in WO2007/058832, which is incorporated by reference in its entirety. In some embodiments, the ITK inhibitor is an ITK inhibitor compound described in WO2004/016610, which is incorporated by reference in its entirety. In some embodiments, the ITK inhibitor is an ITK inhibitor compound described in WO2004/016611, which is incorporated by reference in its entirety. In some embodiments, the ITK inhibitor is an ITK inhibitor compound described in WO2004/016600, which is incorporated by reference in its entirety. In some embodiments, the ITK inhibitor is an ITK inhibitor compound described in WO2004/016615, which is incorporated by reference in its entirety. In some embodiments, the ITK inhibitor is an ITK inhibitor compound described in WO2005/026175, which is incorporated by reference in its entirety. In some embodiments, the ITK inhibitor is an ITK inhibitor compound described in WO2006/065946, which is incorporated by reference in its entirety. In some embodiments, the ITK inhibitor is an ITK inhibitor compound described in WO2007/027594, which is incorporated by reference in its entirety. In some embodiments, the ITK inhibitor is an ITK inhibitor compound described in WO2007/017455, which is incorporated by reference in its entirety. In some embodiments, the ITK inhibitor is an ITK inhibitor compound described in WO2008/025820, which is incorporated by reference in its entirety. In some embodiments, the ITK inhibitor is an ITK inhibitor compound described in WO2008/025821, which is incorporated by reference in its entirety. In some embodiments, the ITK inhibitor is an ITK inhibitor compound described in WO2008/025822, which is incorporated by reference in its entirety. In some embodiments, the ITK inhibitor is an ITK inhibitor compound described in WO2011/017219, which is incorporated by reference in its entirety. In some embodiments, the ITK inhibitor is an ITK inhibitor compound described in WO2011/090760, which is incorporated by reference in its entirety. In some embodiments, the ITK inhibitor is an ITK inhibitor compound described in WO2009/158571, which is incorporated by reference in its entirety. In some embodiments, the ITK inhibitor is an ITK inhibitor compound described in WO2009/051822, which is incorporated by reference in its entirety. In some embodiments, the Itk inhibitor is an Itk inhibitor compound described in US20110281850, which is incorporated by reference in its entirety. In some embodiments, the Itk inhibitor is an Itk inhibitor compound described in WO2014/082085, which is incorporated by reference in its entirety. In some embodiments, the Itk inhibitor is an Itk inhibitor compound described in WO2014/093383, which is incorporated by reference in its entirety. In some embodiments, the Itk inhibitor is an Itk inhibitor compound described in U.S. Pat. No. 8,759,358, which is incorporated by reference in its entirety. In some embodiments, the Itk inhibitor is an Itk inhibitor compound described in WO2014/105958, which is incorporated by reference in its entirety. In some embodiments, the Itk inhibitor is an Itk inhibitor compound described in US2014/0256704, which is incorporated by reference in its entirety. In some embodiments, the Itk inhibitor is an Itk inhibitor compound described in US20140315909, which is incorporated by reference in its entirety. In some embodiments, the Itk inhibitor is an Itk inhibitor compound described in US20140303161, which is incorporated by reference in its entirety. In some embodiments, the Itk inhibitor is an Itk inhibitor compound described in WO2014/145403, which is incorporated by reference in its entirety.

In some embodiments, the ITK inhibitor has a structure selected from:

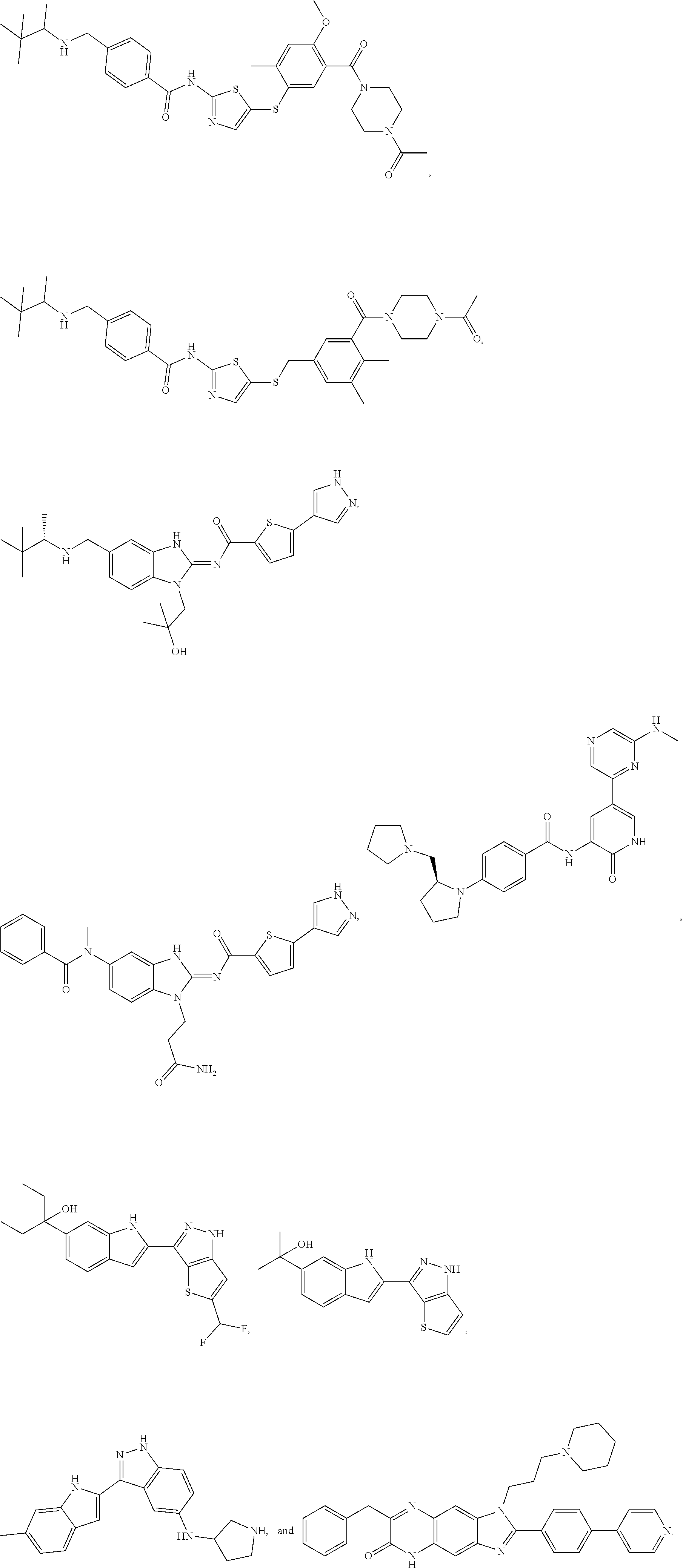

### Pharmaceutical Compositions/Formulations

Disclosed herein, in certain embodiments, are compositions comprising a therapeutically effective amount of an ACK inhibitor compound, and a pharmaceutically acceptable excipient. In some embodiments, the ACK inhibitor compound (e.g., an ITK or BTK inhibitor, such as for example ibrutinib) is a compound of Formula (A). In some embodiments, the ACK inhibitor compound is (R)-1-(3-(4-amino-3-(4-phenoxyphenyl)-1H-pyrazolo[3,4-d]pyrimidin-1-yl)piperidin-1-yl)prop-2-en-1-one (i.e., PCI-32765/ibrutinib).

Pharmaceutical compositions of ACK inhibitor compound (e.g., an ITK or BTK inhibitor, such as for example ibrutinib) are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of an ACK inhibitor compound (e.g., an ITK or BTK inhibitor, such as for example ibrutinib) with other chemical components, such as carriers, stabilizers, diluents, dispersing agents, suspending agents, thickening agents, and/or excipients.

Pharmaceutical compositions are optionally manufactured in a conventional manner, such as, by way of example only, by means of conventional mixing, dissolving, granulating, dragee-making, levigating, emulsifying, encapsulating, entrapping or compression processes.

The pharmaceutical formulations described herein are administered by any suitable administration route, including but not limited to, oral, parenteral (e.g., intravenous, subcutaneous, intramuscular), intranasal, buccal, topical, rectal, or transdermal administration routes.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the compositions are formulated into capsules. In some embodiments, the compositions are formulated into solutions (for example, for IV administration).

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In some embodiments, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In some embodiments, the pharmaceutical compositions are formulated such that the amount of the ACK inhibitor (e.g., an ITK or BTK inhibitor, such as for example ibrutinib) in each unit dosage form is about 140 mg per unit.

### Kits/Articles of Manufacture

Described herein are kits for treating alloantibody driven chronic graft versus host disease (cGVHD) in a patient in need thereof comprising a therapeutically-effective amount of an ACK inhibitor compound (e.g., an ITK or BTK inhibitor, such as for example ibrutinib).

Further described herein are kits for preventing the occurrence of alloantibody driven chronic graft versus host disease (cGVHD) or reducing the severity of alloantibody driven cGVHD occurrence in a patient requiring cell transplantation comprising a therapeutically effective amount of an ACK inhibitor compound (e.g., an ITK or BTK inhibitor, such as for example ibrutinib), wherein a therapeutically effective amount of an ACK inhibitor compound (e.g., an ITK or BTK inhibitor, such as for example ibrutinib) is administered prior to or concurrently with allogeneic hematopoietic stem cells and/or allogeneic T-cells.

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disorder that benefit by inhibition of BTK, or in which BTK is a mediator or contributor to the symptoms or cause.

The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes, carrier, package, container, vial and/or tube labels listing contents and/or instructions for use, and package inserts with instructions for use. A set of instructions will also typically be included.

In some embodiments, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a pharmaceutical composition comprising the ACK inhibitor compound (e.g., an ITK or BTK inhibitor, such as for example ibrutinib) is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

### Example 1

To determine whether ibrutinib could reverse established cGVHD, a murine model of alloantibody driven multi-organ system cGVHD including bronchiolar obliterans (BO) (MHC disparate, C57BL/6→B10.BR) was utilized.

Materials and Methods

Mice: C57BL/6 (H2b) mice were purchased from the National Cancer Institute or from The Jackson Laboratory. B10.BR (H2k) mice were purchased from The Jackson Laboratory. The C57BL/6 XID mouse (kinase activity of BTK is genetically abrogated) was commercially obtained from The Jackson Laboratory and the ITK−/− mouse was a gift. Both strains are maintained on the defined C57BL/6 genetic background. All mice were housed in a pathogen-free facility and used with the approval of the respective institutional animal care committee.

Therapeutic allo-HSCT model: The C57BL/6→B10.BR model has been described previously (Srinivasan, M. et al. Blood 119, 1570-1580 (2012)). In brief, B10.BR recipients conditioned with 120 mg/kg/day I.P. cyclophosphamide (Cy) on days −3 and −2 and 8.3 Gy TBI (using a ¹³⁷Cesium irradiator) on day −1 were engrafted with 1×10⁷ Thy1.2 depleted C57BL/6 derived bone marrow (BM) cells with (or without) 1×10⁶ allogeneic splenocytes.

Therapeutic administration of ibrutinib via drinking water was conducted as previously described (Dubovsky 2013). Mice received a dose equivalent to 15 mg/Kg/day in 0.4% methylcellulose by intraperitoneal injection starting day 28 post-transplant for the C57BL/6→B10.BR model. Cyclosporine A was administered I.P. in 0.2% CMC at 10 mg/kg/day starting at day 25 for 2-weeks followed by 3× weekly (Blazar, B. R. et al. Blood 92, 3949-3959 (1998)).

Pulmonary function tests: Pulmonary function tests (PFTs) were performed on anesthetized mice using whole-body plethysmography with the Flexivent system (SCIREQ).

GC detection: GC detection was conducted using 6 μm spleen cryosections stained using rhodamine peanut agglutinin as previously described (Srinivasan, M. et al. Blood 119, 1570-1580 (2012)).

Masson Trichrome staining: 6 μm cryosections were fixed for 5 minutes in acetone and stained with hematoxylin and eosin to determine pathology and with the Masson's trichrome staining kit (Sigma) for detection of collagen deposition. Histopathology scores were assigned as described (Blazar, B. R. et al. Blood 92, 3949-3959 (1998)). Collagen deposition was quantified on trichrome stained sections as a ratio of area of blue staining to area of total staining using the Adobe Photoshop CS3 analysis tool.

Histopathologicalscoring: Coded pathologic analysis of H&E stained sections was done by a trained veterinary pathologist in an unbiased manner. Scores ranged from 0 to 4 indicating the maximum number of lymphoplasmacytic and histiocytic cellular cuffs infiltrating the surrounding airways or vasculature in 2 different 4× microscopic fields and the number of infiltrating aggregates. 0 cuffs=0, 1 to 5 cuffs=1, 6 to 10 cuffs and <6 aggregates=2, 11 to 15 cuffs and <15 aggregates=3, and >16 cuffs=4. Limited foci of alveolar histiocytosis present with 0 cuffs were considered incidental. For renal H&E stained sections both perivascular lymphoplasmacytic infiltration and intratubular protein were quantified by a trained veterinary pathologist on coded specimens. Scoring ranged from 0 to 4 according to the following guidelines: No inflammatory infiltrates and hyaline eosinophilic material absent from tubular lumens=0, Scattered foci lymphocytes and plasma cells surrounding renal vasculature or <6 tubular profiles containing hyaline eosinophilic material=1, between 1 and 2 aggregates of inflammatory cells <10 cells in diameter or 6 to 10 tubules containing hyaline eosinophilic material=3, between 3 and 4 foci of inflammatory cells which are up to 20 cells in diameter or between 11 and 15 tubules containing hyaline eosinophilic material=3, 5 inflammatory cell foci or more or fewer than 5 which are >20 cells in diameter or >15 tubules containing hyaline eosinophilic material=4.

Statistical analysis: Unless otherwise noted, a two-tailed student's T-test was used for normal data at equal variance. Significance was considered for p<0.05.

Results

Therapeutic Administration of Ibrutinib Ameliorated Pulmonary Fibrosis and the Development of Bronchiolitis Obliterans.

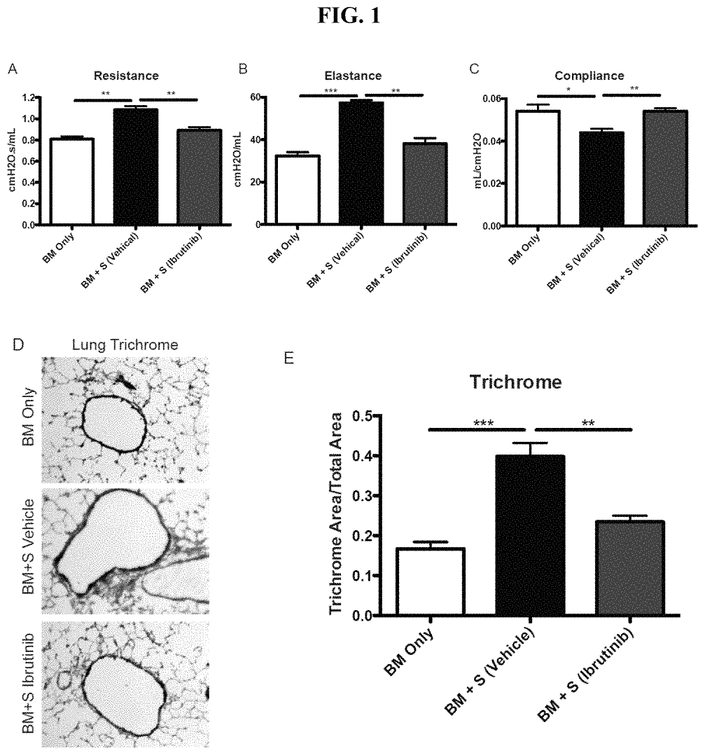

cGVHD is characterized by a wide variety of autoimmune phenomena which are incompletely reacapitulated by any single in vivo animal model. Recently published consensus criterion from the National Institutes of Health considers BO the only pathognomonic manifestation of cGVHD within the lung. The C57BL/6→B10.BR model has been shown to develop multi-organ system disease including BO starting at day 28 post-HSCT. Therapeutic administration of ibrutinib beginning at day 28 and continuing indefinitely curtailed the development of BO in vivo as measured by pulmonary resistance (p=0.0090), elastance (p=0.0019), and compliance (p=0.0071) (FIGS. 1A, B, and C).

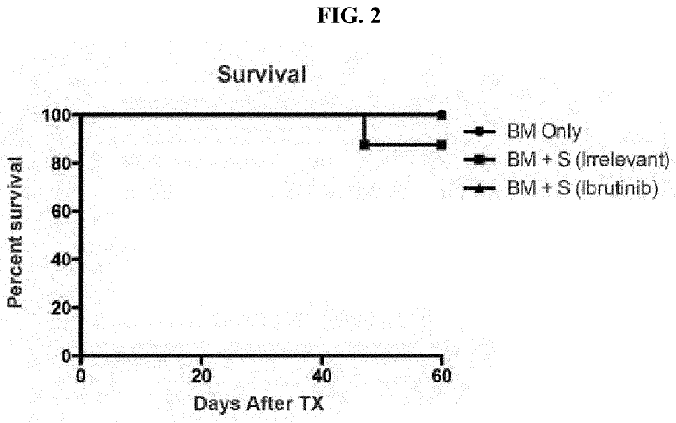

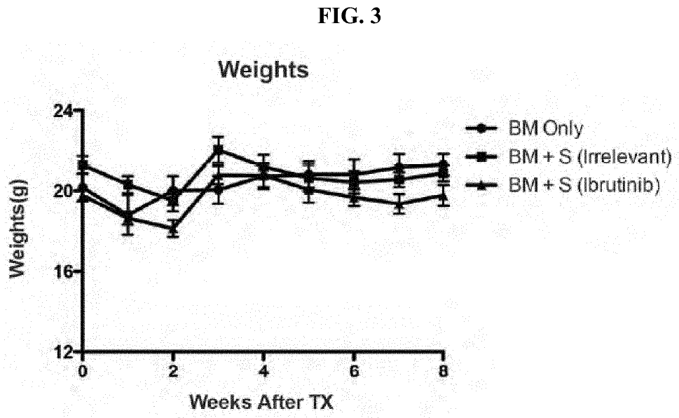

BO is causally related to pulmonary collagen deposition and tissue fibrosis. Masson Trichrome staining of inflated pulmonary tissues from 4 mice derived from 3 experiments revealed less peribroncheolar collagen fibrosis amongst ibrutinib treatment animals (FIG. 1D). Quantified trichrome staining data confirmed that ibrutinib therapy ameliorates pulmonary fibrosis caused by cGVHD (p<0.0001) (FIG. 1E). Death due to cGVHD is rare in this model and indeed 100% survival in the ibrutinib cohort was observed (FIG. 2). Weekly evaluation of mouse bodyweight revealed little variation between groups (FIG. 3). These functional data indicate that ibrutinib therapeutically combats the underlying fibrotic pathogenesis of BO in the C57BL/6→B10.BR cGVHD model.

Ibrutinib Limited In Vivo Germinal Center Reactions and Ig Deposition within Pulmonary Tissues.

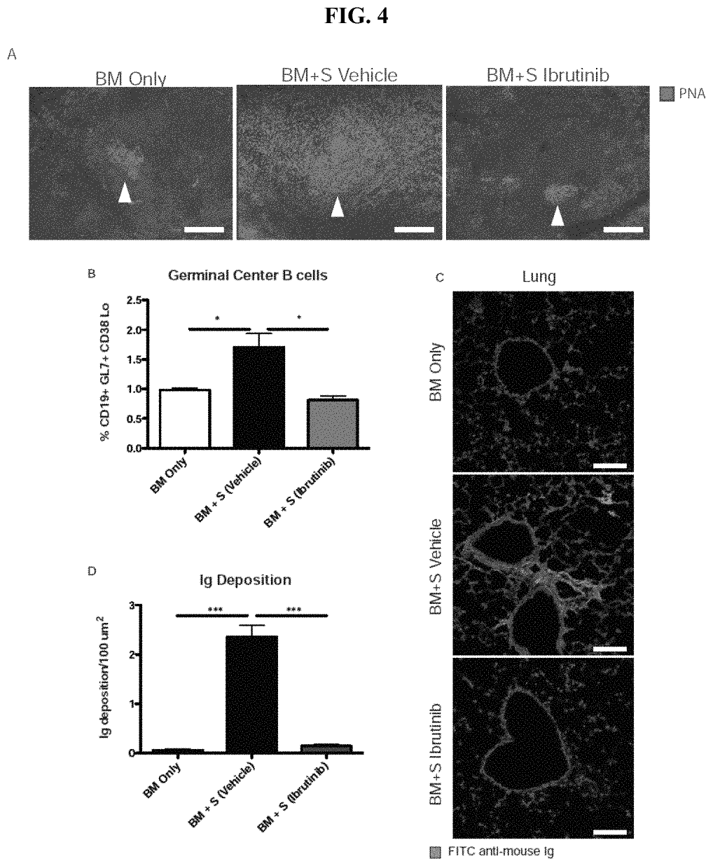

Ibrutinib's ability to block BCR-induced activation of BTK is well defined, however it remains unclear if allo-reactive B-cells in the context of the GC are effectively inhibited. To study this the C57BL/6→B10.BR mouse model, in which robust GC reactions sustain pathogenic alloreactive B-lymphocytes and lead to Ig deposition within the liver and lungs and the development of BO, was utilized. Peanut agglutinin staining revealed GC reactions within the spleen and ibrutinib therapy reduced the overall size, cellularity, and number of GC reactions as compared to vehicle treated mice with active cGVHD (FIG. 4A). On day 60 post-HSCT isolated splenocytes from 8 mice per group were analyzed by flow cytometry for CD19+GL7+CD38lo germinal center B-cells. Data revealed that ibrutinib significantly inhibited the cGVHD-induced formation germinal center B-cells within the spleen (p=0.0222) (FIG. 4B). These results indicated a significant drop in the alloreactive GC reaction which is potentially related to the TEC-kinase blockade caused by ibrutinib.

The functional product of allo-reactive GC B-cells is soluble Ig which deposits within healthy tissues. In the C57BL/6→B10.BR cGVHD model, BO is inextricably related to the deposition of soluble Ig within pulmonary tissues and the fibrotic cascade which this initiates. By blocking B-cell reactivity, ibrutinib limited pulmonary deposition of allo-Ig as quantified at day 60 post-HSCT using immunofluorescent microscopy (FIG. 4C). As expected, quantified immunofluorescent signal revealed significant and complete ablation of pulmonary Ig deposition after therapeutic ibrutinib treatment (p<0.001)(FIG. 4D). These data confirmed that a clinically relevant downstream effect of ibrutinib therapy in the setting of cGVHD is the blockade of Ig deposition within healthy tissues.

Genetic Ablation of BTK or ITK Activity in Allogeneic Donor Cell Engraftment Confirmed that Both TEC-Kinases are Required for the Development of cGVHD.

The XID mouse in which the kinase activity of BTK is genetically abrogated and the ITK−/− mouse have been fully characterized on the C57BL/6 genetic background (Numata et al., Int Immunol 9(1):139-46, 1997; and Liu et al., J Exp Med 187(10):1721-7, 1998). Given ibrutinib's ability to inhibit both ITK and BTK the relative independent contribution of ITK and BTK to the development of cGVHD was examined. To answer this question pulmonary function at day-60 post-HSCT was examined, as this represents a primary functional measurement of cGVHD induced lung injury and fibrosis in the C57BL/6→B10.BR model.

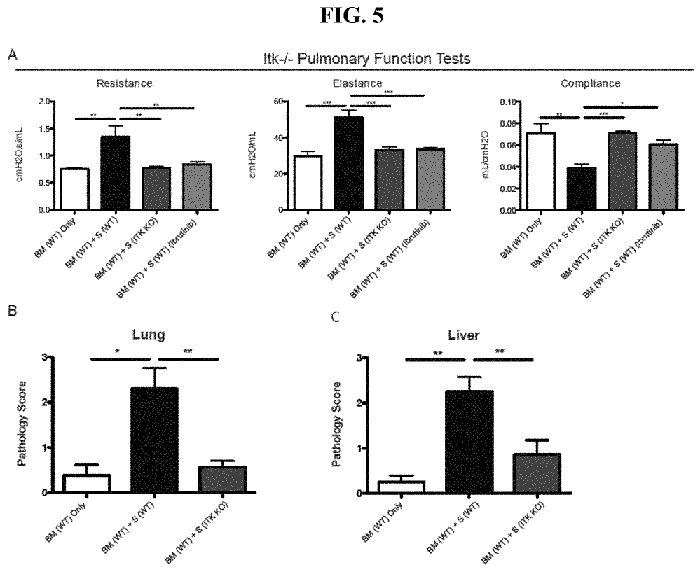

cGVHD sustaining T-cells in this model originate from mature lymphocytes incorporated into the donor cell engraftment. To recapitulate the effect of ITK inhibition within these cGVHD causative T-lymphocytes, ITK−/− splenic T-cells along with wild type BM were engrafted into allogeneic recipients. Day 60 pulmonary function tests including resistance, elastance, and compliance were uniformly and significantly (p=0.0014; p=0.0028; p=0.0003) restored to healthy levels in mice receiving ITK−/− splenic T-cells as part of their engraftment, when compared to mice receiving wild type splenic T-cells (FIG. 5). These data revealed that T-cell ITK activity was necessary for the development of cGVHD.

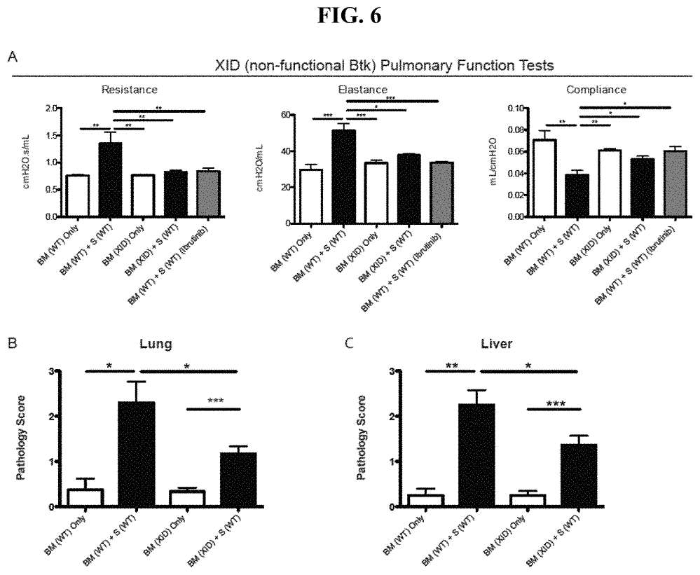

cGVHD pathogenic B-cells arise from the ontogeny of donor hematopoietic stem cells; therefore XID BM along with wild type splenic T-cells were engrafted to recapitulate BTK inhibition in all allogeneic-derived B-cells. Pulmonary function tests conducted at day 60 post-HSCT revealed that BTK activity was essential to the development of BO (FIG. 6). Pulmonary metrics of resistance, elastance, and compliance were significantly improved (p=0.0025; p=0.0025; p=0.0496) in mice receiving XID BM, as compared to mice receiving wild type bone marrow.

In summary, in the C57BL/6→B10.BR cGVHD model, ibrutinib restored pulmonary function, abated germinal center reactions and tissue immunoglobulin deposition, and reversed lung and liver fibrosis. Our analysis revealed that ibrutinib therapeutically blocked allo-reactive germinal center (GC) B-cells, immunoglobulin (Ig) deposition, and lung fibrosis associated with the progression of cGVHD.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating alloantibody driven chronic graft versus host disease (cGVHD) in a patient, comprising administering to a patient in need thereof a therapeutically effective amount of a compound of Formula (A) having the structure: 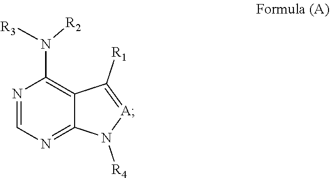
wherein:
A is N;
R₁ is phenyl-O-phenyl or phenyl-S-phenyl;
R₂ and R₃ are independently H;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;
G is 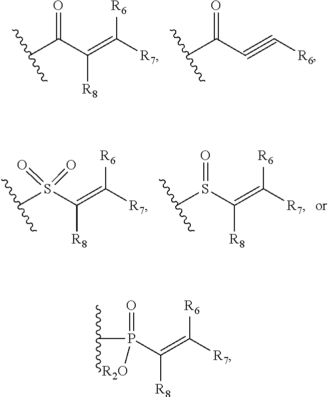
wherein,
R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or substituted or unsubstituted alkyl; or a pharmaceutically acceptable salt thereof, thereby treating the GVHD in the patient.

2-20. (canceled)


SEQUENCE LISTING (1 sequence)

SEQ ID NO: 1 - protein, 15 aa, Artificial Sequence
  Synthetic polypeptide
AVLESEEELYSSARQ